SEQUENCE LISTING (20 sequences)

SEQ ID NO: 1 - DNA, 20 bp, Artificial Sequence
  Synthetic
cagaagcacacattgaagaa

SEQ ID NO: 2 - DNA, 20 bp, Artificial Sequence
  Synthetic
tgtaagtagccagaggaagg

SEQ ID NO: 3 - DNA, 20 bp, Artificial Sequence
  Synthetic
gggaaagcagaggaaatcaa

SEQ ID NO: 4 - DNA, 20 bp, Artificial Sequence
  Synthetic
tgaaagttgccatacagaag

SEQ ID NO: 5 - DNA, 21 bp, Artificial Sequence
  Synthetic
gccgttacagggaaatactgg

SEQ ID NO: 6 - DNA, 18 bp, Artificial Sequence
  Synthetic
cctcaacatcggggctct

SEQ ID NO: 7 - DNA, 20 bp, Artificial Sequence
  Synthetic
ggatgcctgggagagaatcg

SEQ ID NO: 8 - DNA, 20 bp, Artificial Sequence
  Synthetic
tcgcctgctcttcgaaactg

SEQ ID NO: 9 - DNA, 20 bp, Artificial Sequence
  Synthetic
aacaggctggtttggagaag

SEQ ID NO: 10 - DNA, 20 bp, Artificial Sequence
  Synthetic
tgccattgctcactatgctc

SEQ ID NO: 11 - DNA, 20 bp, Artificial Sequence
  Synthetic
gccaggagcaagaccgtgtt

SEQ ID NO: 12 - DNA, 19 bp, Artificial Sequence
  Synthetic
tgccccaccactgcctgta

SEQ ID NO: 13 - DNA, 20 bp, Artificial Sequence
  Synthetic
tgggggacacaggctctaca

SEQ ID NO: 14 - DNA, 22 bp, Artificial Sequence
  Synthetic
gcctgcaggatggaactctcaa

SEQ ID NO: 15 - DNA, 20 bp, Artificial Sequence
  Synthetic
cctacagggcggacttcaag

SEQ ID NO: 16 - DNA, 22 bp, Artificial Sequence
  Synthetic
ggatggcaaaggcagtgtaact

SEQ ID NO: 17 - DNA, 20 bp, Artificial Sequence
  Synthetic
gaagtcacgaagctgaacca

SEQ ID NO: 18 - DNA, 22 bp, Artificial Sequence
  Synthetic
cctgcactgtgctagaagtctc

SEQ ID NO: 19 - DNA, 20 bp, Artificial Sequence
  Synthetic
gatgctccccgggctgtatt

SEQ ID NO: 20 - DNA, 20 bp, Artificial Sequence
  Synthetic
ggggtacttcagggtcagga

(19) United States
(12) Patent Application Publication
(10) Pub. No.: US 2023/0372407 A1
(43) Pub. Date: Nov. 23, 2023

(54) METHODS FOR MODULATING TLR4 SIGNALING

(71) Applicant: President and Fellows of Harvard College, Cambridge, MA (US)

(72) Inventors: Dennis L. Kasper, Cambridge, MA (US); Kailyn L. Stefan, Cambridge, MA (US)

(73) Assignee: President and Fellows of Harvard College, Cambridge, MA (US)

(21) Appl. No.: 18/010,427

(22) PCT Filed: Jun. 15, 2021
(86) PCT No.: PCT/US2021/037310

§ 371 Date: Dec. 14, 2022

Related U.S. Application Data
(60) Provisional application No. 63/085,392, filed on Sep. 30, 2020.
(60) Provisional application No. 63/039,843, filed on Jun. 16, 2020.
(60) Provisional application No. 63/039,432, filed on Jun. 15, 2020.

(51) Int. Cl. A61K 35/74; A61P 31/12; A61K 45/06

(52) U.S. Cl. CPC A61K 35/74; A61P 31/12; A61K 45/06

(57) ABSTRACT

Provided herein are methods of use involving Bacteroides glycolipid compositions.

### RELATED APPLICATIONS

This application claims the benefit of the filing dates of U.S. Provisional Application Nos. 63/039432 filed on Jun. 15, 2020, 63/039,843 filed on Jun. 16, 2020, and 63/085,392 filed on Sep. 30, 2020, the entire contents of which are incorporated by reference herein.

### FEDERALLY SPONSORED RESEARCH

This invention was made with Government support under W81XWH1910625 awarded by the Department of Defense and AI120269 awarded by the National Institutes of Health. The Government has certain rights in the invention.

### BACKGROUND OF INVENTION

The human body, like that of other mammals, is colonized by trillions of microbial organisms, including bacteria, viruses, fungi, and archaea, collectively referred to as the commensal microbiota. (Erturk-Hasdemir and Kasper, 2013; Human Microbiome Project, 2012) With advances in culture-independent DNA sequencing technologies and bioinformatics, there has been a rapid increase in our knowledge of the diversity and dynamics of the microbiota over the past two decades. (Human Microbiome Project, 2012; Qin et al., 2010) Current estimates suggest that there are roughly as many bacterial cells as human cells in the body, and approximately a hundred times more bacterial genes than human genes, the vast majority of which reside in the lower gastrointestinal (GI) tract, which is colonized by an estimated 10¹⁴ bacteria. (Qin et al., 2010; Savage, 1977; Sender et al., 2016; Turnbaugh et al., 2007) Despite these advances, relatively little is understood about the molecular mechanisms by which specific commensal microbes interact with their mammalian host.

### SUMMARY OF INVENTION

This disclosure is premised in part on the finding that Bacteroides spp. in the gut microbiome mediate a protective immune response through the TLR4 signaling pathway. This finding is surprising, in part, because it has been previously shown that the TLR4 pathway also mediates aberrant immune responses that occur in response to other bacterial species. For example, lipopolysaccharide (LPS) from E. coli also mediates its effects through engagement of TLR4 but the consequences of that engagement, such as for example sepsis, are detrimental to the host.

This disclosure further identifies that Bacteroides spp. engage TLR4 and trigger a protective immune response through the glycolipid moiety that anchors polysaccharide A (PSA) to the bacterial membrane. The downstream cascade that occurs following such TLR4 engagement by Bacteroides spp. and their components, and subsequent signaling, was also unexpected and unpredictable.

The disclosure therefore contemplates the use of the Bacteroides glycolipid in a variety of forms including an isolated form, as lipidated PSA, as an outer membrane derived from a Bacteroides sp., or as whole, inactivated Bacteroides spp., as examples.

These findings indicate that the Bacteroides glycolipid may be used in a number of clinical applications. For example, the Bacteroides glycolipid may be used in the treatment of TLR4-dependent conditions, in order to suppress or down-regulate TLR4 engagement and/or signaling. Such conditions may be currently treated (or contemplated to be treated) using a TLR4 antagonist. The Bacteroides glycolipid of this disclosure may function by competing for binding to TLR4 and redirecting the subsequent TLR4 signaling cascade towards a beneficial immune response, rather than blocking TLR4 signaling altogether. Thus, the Bacteroides glycolipid may be advantageous over previously described TLR4 antagonists in the management of TLR4-dependent conditions.

More generally, the Bacteroides glycolipid is contemplated for use in modulating TLR4 engagement and/or signaling in subjects in need thereof. Such subjects include those receiving or who have received a TLR4 agonist or antagonist. The Bacteroides glycolipid may act in these situations to modulate adverse effects of the TLR4 agonist or antagonist.

The Bacteroides glycolipid has also been shown to provide protective immunity, particularly to viral infections, in subjects who are experiencing or who are likely to experience an imbalance in the gut microbiome. Such subjects may be those who have been or will be administered a broad-spectrum antibiotic that will adversely impact the gut microbiome. Such subjects may be more susceptible to other infections such as for example viral infection. The Bacteroides glycolipid may be used in these situations in order to protect the subject from such infections during the course of treatment with the broad-spectrum antibiotic. A similar situation may present itself in subjects undergoing cancer therapy, and therefore such subjects are also contemplated for treatment with the Bacteroides glycolipid.

Thus the Bacteroides glycolipid may be used to enhance the immune response of a subject in order to provide such subject protection against a subsequent (or ongoing) viral exposure. The Bacteroides glycolipid has been shown to provide protection against two diverse viruses of different classes, and is contemplated for use as a broad spectrum anti-viral agent.

This disclosure therefore provides a method for stimulating an anti-viral immune response in a subject, comprising administering to the subject an effective amount of a Bacteroides glycolipid comprising a diglucosamine substituted with one or more acyl chains. The glycolipid may be administered on a regular basis. The immune response protects the subject against a viral infection resulting from exposure to a virus.

Also provided herein is a method for treating a subject to protect against a viral infection resulting from exposure to a virus, comprising administering to the subject an effective amount of a Bacteroides glycolipid comprising a diglucosamine substituted with one or more acyl chains, on a regular basis.

Also provided herein is a method for treating a subject, comprising administering an effective amount of a Bacteroides glycolipid comprising a diglucosamine substituted with one or more acyl chains, on a regular basis, to a subject at risk of exposure to a virus or developing a viral infection.

The glycolipid may be derived from B. fragilis or another Bacteroides spp. or it may be produced de novo in a cell free system. It may be provided in any one or a combination of a variety of forms, as provided herein.

In some embodiments, the glycolipid is administered on a regular basis. In some embodiments, a regular basis is daily, every two days, every 3 days, every 4 days, every 5 days, every 6 days, weekly, every two weeks, or monthly.

In some embodiments, the subject is immunocompromised.

In some embodiments, the subject was recently treated with an antibiotic, optionally an antibiotic that targets anaerobic bacteria, or an antibiotic that is a broad spectrum antibiotic. In some embodiments, the subject was treated with the antibiotic in the last 2 months, in the last 1 month, or in the last week. In some embodiments, the subject is being treated with an antibiotic, optionally an antibiotic that targets anaerobic bacteria. In some embodiments, the subject will be treated with an antibiotic, optionally an antibiotic that targets anaerobic bacteria. In some embodiments, the subject will be treated with an antibiotic in the next day, in the next week, or in the next month.

In some embodiments, the subject has come into contact with a subject exposed to the virus or having the viral infection or with a bodily fluid of a subject exposed to the virus or having the viral infection. In some embodiments, the subject is present or will be present in a region in which the viral infection is endemic.

In some embodiments, the viral infection is selected from the group consisting of SARS coronavirus, influenza virus, Ebola virus, norovirus, Zika virus, West Nile virus, Dengue virus, herpesvirus, equine encephalitis virus, respiratory syncytial virus (RSV), cytomegalovirus, rabies virus, or measles virus infection.

In some embodiments, the Bacteroides glycolipid is administered orally, optionally in an enteric-coated pill, capsule or tablet.

In some embodiments, the Bacteroides glycolipid is provided in an unconjugated, cell-free and membrane-free form. In some embodiments, the Bacteroides glycolipid is provided as lipidated PSA. In some embodiments, the Bacteroides glycolipid is provided as Bacteroides outer membrane vesicle (OMV). In some embodiments, the Bacteroides glycolipid is provided as inactivated Bacteroides spp. In some embodiments, the Bacteroides glycolipid is provided as live attenuated Bacteroides spp. In some embodiments, the Bacteroides glycolipid is provided as live unattenuated Bacteroides spp.

In some embodiments, the glycolipid is not administered with a viral antigen. In some embodiments, the glycolipid is administered with a viral antigen.

Also provided is a method for modulating TLR4 signaling comprising administering to a subject in need thereof an effective amount of a Bacteroides glycolipid comprising a diglucosamine substituted with one or more acyl chains.

In some embodiments, the subject has a TLR4-mediated condition.

In some embodiments, the subject does not have sepsis.

In some embodiments, the subject is administered or exposed to a TLR4 agonist. In some embodiments, the TLR4 agonist is buprenorphine, carbamazepine, fentanyl, levorphanol, lipopolysaccharide (LPS), methadone, morphine, oxycarbezepine, oxycodone, pethidine, tapentadol, or morphine-3-glucuronide.

In some embodiments, the subject is administered or exposed to a TLR4 antagonist. In some embodiments, the TLR4 antagonist is naloxone, naltrexone, propentofylline, palmitoylethanolamide, amitriptyline, cyclobenzaprine, ketotifen, imipramine, mianserin, ibudilast, pinocembrin, or resatorvid.

These and other aspects and embodiments will be described in greater detail herein.

### BRIEF DESCRIPTION OF DRAWINGS

It is to be understood that the Figures are not necessarily to scale, emphasis instead being placed upon generally illustrating the various concepts discussed herein.

FIGS. 1A-1D. Microbiota depletion reduces local and systemic ISG expression. Mesenteric lymph nodes (mLNs) and spleens were harvested from age- and gender-matched C57Bl/6 WT or Ifnb1^{-/-} SPF mice with and without broad-spectrum antibiotics (ABX) treatment. RNA was isolated from whole tissue samples followed by qRT-PCR to analyze ISG expression levels. Fold change gene expression in the mLN (FIG. 1A, FIGS. 1C-1D) or spleen (FIGS. 1B, 1C-1D) was calculated compared to WT SPF mice using the ΔΔCT method, with ActB as the reference gene and depicted as a heat map of all ISGs tested (FIGS. 1A, 1B) or representative bar graphs of mean +/− SEM with each point representing one mouse for Ifit3 (FIG. 1C) and Oasl2 (FIG. 1D). One-way ANOVA statistical analysis followed by Tukey's multiple comparisons test. Details of statistical analyses can be found in Tables 1-2. ns=not significant, ****p<0.0001.

FIGS. 2A-2H. The commensal microbiota regulates IFNβ expression by dendritic cells in the colon LP. FIGS. 2A-2D. Single cell suspensions were prepared from spleens, mLNs, and colon LP of age-matched SPF or ABX treated IFNβ-YFP reporter mice and analyzed by flow cytometry. FIG. 2A shows representative dot plots of IFNβ-YFP+CD45⁺ cells in each tissue. FIG. 2B shows the frequency of IFNβ-YFP+ cells out of CD45⁺ cells (colon LP N=12, mLN N=13, spleen N=13). FIG. 2C shows the frequency of CD11c⁺ DCs out of total IFNβ-YFP⁺CD45⁺ cells in the colon LP (N=10). FIG. 2D shows the frequency of IFNβ-YFP⁺CD11c⁺ cells out of CD45⁺ cells (ABX N=7, SPF N=10). FIGS. 2E-2G. Dendritic cells were isolated from single cell suspensions of different tissues from WT GF and WT SPF mice, yielding dendritic cell positive (DC⁺) and negative (DC⁻) fractions, and Ifnb1 expression was analyzed by qRT-PCR. Relative expression of Ifnb1 in the whole tissue (N=10), DC⁺ (N=9), and DC⁻ (N=6) fractions of the colon LP (FIG. 2E), the DC⁺ fraction of the colon LP (N=5), mLN (N=6), or spleen (N=3) (FIG. 2F) or in colon LP DC⁺ cells from WT SPF (N=6) or WT GF mice (N=6) (FIG. 2G). Fold change gene expression was calculated using the ΔΔCT method, with Actb as the reference gene, compared to the DC⁺ (FIG. 2E), colon LP (FIG. 2F), or SPF samples (FIG. 2G). (H) Frequency of CD11b+CD103−, CD11b+CD103+, and CD11b-CD103+DC subsets out of IFNβ-YFP⁺CD11c+ colonic LP dendritic cells. Bars represent mean +/− SEM. Statistical analysis with (B,E,F) one-way ANOVA followed by Tukey's multiple comparisons test and (D,G) unpaired t-test. N=number of mice, N.D.=not detected, *p<0.05, **p<0.01, ***p<0.001, ****p<0.0001, ns=not significant.

FIGS. 3A-3F. B. fragilis polysaccharide A (PSA) induces IFNβ in vitro and in vivo. FIG. 3A. WT germ free mice were gavaged with vehicle (GF, N=5) or colonized at 4 weeks of age with B. fragilis (N=5) or C. ramosum (N=4). Two weeks post colonization, dendritic cells were isolated from colon LP single cell suspensions, RNA was isolated, and Ifnb1 expression was analyzed by qRT-PCR. Fold change gene expression to GF was calculated using the ΔΔCT method with ActB as a reference gene. FIG. 3B-3E. BMDCs were cultured from WT, Ifnb1^{-/-}, or Ifnar1^{-/-} mice. FIG. 3B. IFNβ levels in the supernatants of WT BMDCs treated with vehicle control (CTRL, N=6) or 100 ug/mL B. fragilis OM extract (N=3). FIG. 3C. ELISA analysis of IFNβ levels in the supernatants of WT BMDCs treated with 50 ug/mL PSA for 0 (N=15), 6 (N=15), or 24 hrs (N=6). FIG. 3D. RNA was isolated from WT BMDCs 24 hrs post treatment with vehicle control (CTRL, N=4) or 50 ug/mL PSA (N=3) and ISG expression was analyzed by qRT-PCR. Fold change gene expression to CTRL was calculated using the ΔΔCT method with Actb as the reference gene. FIG. 3E. After 24 hrs of treatment with 100 ug/mL PSA, BST2 mean fluorescence intensity (MFI) gated on live CD11c⁺ cells was measured by flow cytometry (N=4 for each condition). FIG. 3F. WT SPF mice were gavaged with 150 ug PSA and after 1.5 hrs Ifnb1 expression by colon LP DCs was analyzed by qRT-PCR (SPF, N=8; SPF+PSA, N=3). Fold change gene expression to SPF was calculated using the ΔΔCT method with Actb as the reference gene. Data represents average +/− SEM. Statistical analysis with one-way ANOVA followed by Dunnett's multiple comparisons test (FIGS. 3A-3C), unpaired t-test (FIGS. 3B, 3D, 3F) and Two-way ANOVA followed by Sidak's multiple comparisons test to WT (FIG. 3E). N.D.=not detected, ns=not significant, *p<0.05, **p<0.01, ***p<0.001, ****p<0.0001.

FIGS. 4A-4F. Bacteroides outer membrane glycolipids signal through TLR4 to induce IFNβ. FIG. 4A. BMDCs were differentiated from WT (N=27), Tlr2^{-/-} (N=15), Clec7a^{-/-} (N=3), Tlr4^{-/-} (N=12), Trif^{-/-} (N=6), or Myd88^{-/-} (N=5) mice and treated with 50 ug/mL B. fragilis PSA for 6 hrs. IFNβ concentration in the supernatants was measured by ELISA and normalized by subtracting the vehicle control. FIG. 4B. ELISA analysis of IFNβ in the supernatants of WT BMDCs treated for 6 hrs with a dose response of PSA (N=9), the lipooligosaccharide domain of PSA (LOS, N=14), or delipidated PSA (PSA-delipidated, N=3, ND for all concentrations). ELISA analysis of IFNβ in the supernatants of WT or Tlr4^{-/-} BMDCs treated for 6 hrs with vehicle control (CTRL, N=8) or 1 ug/mL LOS (N=8) (FIG. 4C) or 100 ug/mL Bacteroides outer membrane extracts (N=3 per condition) (FIG. 4D). FIG. 4A-4D. Data represents average +/− SEM. (FIGS. 4E and 4F) RNA isolation and qRT-PCR analysis of ISG expression was performed on mLNs and spleens harvested from WT SPF mice, WT SPF mice after 1 week of metronidazole (Met) treatment, or from Tlr4^{-/-} SPF mice. Fold change gene expression in the mLN (FIG. 4E) or spleen (FIG. 4F) was calculated compared to WT SPF mice using the ΔΔCT method, with ActB as the reference gene and depicted as a heat map. Statistical analysis with one-way ANOVA followed by Dunnett's multiple comparisons test to WT (FIG. 4A), two-way ANOVA followed by Dunnett's multiple comparisons test to 0 ug/mL CTRL (FIGS. 4B, 4D), and unpaired t-test (FIG. 4C). FIGS. 4E, 4F. Details of statistical analyses can be found in Tables 3-4. ND=not detected, ns=not significant, *p<0.05, **p<0.01, ***p<0.001, ****p<0.0001.

FIGS. 5A-5D. IFNβ is required for protection from murine vesicular stomatitis virus infection. Littermate Ifnb1^{+/+} (N=19) or Ifnb1^{-/-} (N=12) mice were infected with 10⁶ PFU of VSV strain Indiana by subcutaneous injection into the footpad. Mice were monitored daily for paralysis score (FIG. 5A), percentage of mice with paralysis (incidence of disease) (FIG. 5B), and survival (FIG. 5C). FIG. 5D. The sum of the daily disease scores for each mouse for the duration of the experiment (cumulative disease score) was calculated 14 d.p.i. Statistical analysis with linear regression analysis and unpaired t-test (for each day) (FIG. 5A), log-rank test (FIGS. 5B, 5C), and unpaired t-test (FIG. 5D). *p<0.05, **p<0.01, ***p<0.001.

FIGS. 6A-6E. Microbiota-induced IFNβ enhances resistance to VSV infection. Age- and gender-matched WT or Ifnb1−/− SPF mice were treated with vehicle control or broad spectrum antibiotics (ABX) for 7 days prior to infection as well as vehicle or 75 ug PSA daily starting 4 days before until the day of infection with 106 PFU VSV strain Indiana. Mice were observed daily for daily paralysis score based on ascending paralysis (FIGS. 6A, 6B) and percentage of mice with paralysis (incidence of disease) (FIGS. 6C, 6D). FIG. 6E. The sum of the daily disease scores for each mouse for the duration of the experiment (cumulative disease score) was calculated 14 d.p.i. Statistical analysis with linear regression analysis (FIGS. 6A, 6B), log-rank test (FIGS. 6C, 6D), and one-way ANOVA (FIG. 6E). ns=not significant, *p<0.05, ****p<0.001, ****p<0.0001.

FIGS. 7A-7F. B. fragilis PSA reduces virus infection of BMDCs by signaling through TLR4 to induce IFNβ. BMDCs were infected with VSV-GFP (FIGS. 7A, 7C, 7E) or IAV/PR8-GFP (FIGS. 7B, 7D, 7F) (both MOI=1). GFP⁺ virus-infected cells were analyzed by flow cytometry 24 h.p.i. in WT, Ifnb1^{-/-}, or Tlr4^{-/-} BMDCs primed for 24 hours prior to infection with a dose response of PSA (FIGS. 7A-7D) or 10 ug/mL PSA (FIGS. 7E-7F). Data represents mean +/− SEM. Statistical analysis with unpaired t-test to 0 ug/mL CTRL. VSV-GFP N=3-6 samples per condition. IAV/PR8-GFP N=3-12 samples per condition. ns=not significant, *p<0.05. **p<0.01, ***p<0.001, ****p<0.0001.

FIGS. 8A-8B. Microbiota depletion reduces local and systemic ISG expression. mLNs and spleens were harvested from age- and gender-matched WTSPF or GF mice. RNA was isolated from whole tissue samples followed by qRT-PCR to analyze ISG expression levels. Fold change gene expression in the mLN (FIG. 8A) or spleen (FIG. 8B) was calculated compared to WT SPF mice using the delta-delta CT method, with ActB as the reference gene and depicted. Bar graphs represent mean +/− SEM. Statistical analysis for each gene with unpaired t-test. ns=not significant, *p<0.05, **p<0.01, ***p<0.001.

FIGS. 9A-9C. The commensal microbiota regulates Ifnb1 expression by dendritic cells in the colon LP and mLN. FIG. 9A-9B. Single cell suspensions were prepared from mLNs and spleens harvested from WT SPF mice. Dendritic cell isolation was performed, yielding dendritic cell positive (DC+) and dendritic cell negative (DC−) fractions. Ifnb1 expression was analyzed by qRT-PCR. Relative expression of Ifnb1 normalized to Actb in the mLN (FIG. 9A) and spleen (FIG. 9B) of WT SPF mice. FIG. 9C. ELISA analysis of IFNβ in the serum of WT SPF or antibiotics treated mice (SPF+ABX). Bars represent average +/− SEM. N.D.=not detected.

FIGS. 10A-10C. Inhibition of IFNβ prior to infection increases susceptibility to vesicular stomatitis virus infection. Age- and gender-matched WT SPF mice were treated with 250 ug anti-IFNβ or mouse IgG2a isotype control antibody by intraperitoneal injection 72 and 24 hours prior to subcutaneous infection with 106 PFU VSV strain Indiana. Mice were monitored daily for paralysis score (FIG. 10A) and percentage of mice with paralysis (incidence of disease) (FIG. 10C). FIG. 10C. The sum of the daily disease scores for each mouse for the duration of the experiment (cumulative disease score) was calculated 14 d.p.i. Statistical analysis with linear regression analysis (FIG. 10A), unpaired t-test (FIG. 10B), and log-rank test (FIG. 10C). ns=not significant, *p<0.05, ***p<0.001.

FIGS. 11A-11B. BMDC virus infection representative flow cytometric data. BMDCs were infected with VSV-GFP (FIG. 11A) or IAV/PR8-GFP (FIG. 11B) (both MOI=1). Infection was analyzed by flow cytometry 24 h.p.i. Cells were first gated on viability, followed by analysis of GFP+ virus-infected cells.

FIG. 12. Human specimens respond to PSA in dose-dependent manner.

FIG. 13. PSA induces Tregs in cells obtained from human gut tissue biopsies. Total cLP lymphocytes from 8-10 human colon biopsies were treated with PBS, WT-OMV, DPSA-OMV, or purified PSA for 6-10 days. IL-10 production by CD4+FOXP3+ Tregs was assessed by flow cytometry and intracellular cytokine staining. ** p<0.01, One-way ANOVA. Error bars represent mean +S.E.M.

The color versions of the Figures are available in the file wrappers of the priority applications 63/039432 filed on Jun. 15, 2020, 63/039,843 filed on Jun. 16, 2020, and 63/085,392 filed on Sep. 30, 2020, the entire contents of which are incorporated by reference herein.

### DETAILED DESCRIPTION OF INVENTION

This disclosure is premised in part on the finding that Bacteroides spp. in the gut microbiome mediate a protective immune response through a glycolipid that engages the TLR4 signaling pathway. The glycolipid anchors polysaccharide A (PSA) to the bacterial membrane and its structure is described below. For the sake of clarity and brevity, it is referred to herein as the Bacteroides glycolipid although this does not intend that it must be produced or sourced solely from Bacteroides spp. Rather, this disclosure contemplates that the glycolipid may be generated de novo, apart from naturally occurring sources.

### Glycolipid Compositions

The Bacteroides glycolipid component minimally comprises a diglucosamine, substituted with one or more acyl chains, preferably 3, 4 or 5 acyl chains. Exemplary glycolipid structures are provided in published PCT Application No. WO2018/014012. The diglucosamine moiety may be monophosphorylated or non-phosphorylated (e.g., dephosphorylated). The various teachings provided herein, including aspects and embodiments of the invention, that are described with respect to the glycolipid are to be understood to apply equally to lipooligosaccharide (LOS, lipidated diglucosamine conjugated to an oligosaccharide, as described herein), as well as other compounds or compositions that comprise the glycolipid, including but not limited to lipidated PSA (polysaccharide A conjugated to LOS), outer membranes (OM), outer membrane vesicles (OMV), Bacteroides cells whether live or inactivated or attenuated, and the like, unless stated otherwise.

The diglucosamine may be conjugated to one or more acyl chains, including three, four, or five acyl chains in some instances via for example ester or amide linkages, and thus may be referred to as “0” substituted (or O-linked) or “N” substituted (or N-linked) respectively. In some instances, the glycolipid comprises three, four or five acyl chains. Accordingly, the glycolipid may be referred to herein as tri-acylated, tetra-acylated or penta-acylated forms.

The acyl chains may range in length from 14 to 17 carbons, in some instances. The acyl chains may be unmodified or they may be modified. If modified, the acyl chains may be hydroxy-modified. Thus, in some instances, the glycolipid may comprise one or more acyl chains characterized as C14:0, C14:0-OH, C15:0, C15:0-OH, C16:0, C16:0-OH, C17:0, and C17:0-OH. Any of the acyl chains may be branched at C13-C17.

A single preparation of glycolipid may yield a number glycolipid species that differ from each other with respect to acyl chain number. The exact composition of a glycolipid may be determined using mass spectrometry (MS), wherein different glycolipid species give rise to different and discernable spectra.

Examples of different penta-acylated species include:
(1) one chain of C16:0-OH, three chains of C17:0-OH, and one chain of C15:0,
(2) two chains of C16:0-OH, two chains of C17:0-OH, and one chain of C15:0,
(3) three chains of C16:0-OH, one chain of C17:0-OH, and one chain of C15:0,
(4) four chains of C16:0-OH, and one chain of C15:0, and
(5) four chains of C16:0-OH, and one chain of C14:0.

Various species of tetra-acylated and tri-acylated species are similarly contemplated.

It will therefore be appreciated glycolipids of this disclosure, whether isolated from B. fragilis or generated de novo, and whether conjugated or unconjugated to an oligosaccharide core unit, may comprise any of the foregoing combinations of acyl chains, without limitation:

(1) C16:0-OH acyl chain(s) only,
(2) C17:0-OH acyl chain(s) only,
(3) C16:0-OH and C17:0-OH chain(s) only,
(4) C16:0-OH and C17:0-OH and C15:0 chain(s) only,
(5) C16:0-OH and C17:0-OH and C14:0 chain(s).

The number of each type of chain may vary, and may include without limitation the following options
(1) 0-4 C16:0-OH chains,
(2) 0-4 C17:0-OH chains,
(3) 0 or 1 C14:0 chains, and
(4) 0 or 1 C15:0 chains.

The foregoing examples are not to be considered limiting, and rather the disclosure contemplates various combinations, and combinations of the foregoing, to be used in compositions provided herein.

The disclosure provides compositions comprising glycolipids that are only or predominantly (e.g., greater than 50%, or at least 55%, at least 60%, at least 65%, at least 70%, at least 75%, at least 80%, at least 85%, at least 90%, or at least 95%) tri-acylated, or tetra-acylated, or penta-acylated, or some combination thereof including but not limited to tetra- and penta-acylated.

The preparations provided herein can be characterized by their content of glycolipids, including those released from the tetrasaccharide repeating units of PSA or the oligosaccharide core unit of PSA. Such content can be about at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more (weight by weight of the glycolipid to the remaining components in the preparation). The amount of glycolipid may be determined for example using the gel electrophoresis methods or mass spec methods.

The purity of the glycolipid may also be at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, or 99.9%, or higher (weight by weight of the glycolipid to other Bacteroides-derived components in the preparation, wherein such components are unconjugated to the glycolipid).

Isolated intends that the glycolipid (or other agent) is provided in a more pure form or a more concentrated form compared to its synthesized or naturally occurring form. An “isolated” glycolipid may be a glycolipid that is prepared or obtained from a Bacteroides cell, and is physically separated from a natural environment (e.g., a Bacteroides cell, components of the Bacteroides cell, and/or components of the Bacteroides cell capsular complex such as but not limited to PSB).

In some embodiments, the compositions are substantially free of naturally occurring contaminants such as nucleic acids (e.g., DNA and RNA), proteins, and other components of a Bacteroides cell and/or a Bacteroides capsule. Substantially free, as used herein, intends that these contaminants represent about or less than 5%, less than 1%, less than 0.5%, or less than 0.1% (or less) by weight (weight of the contaminant to weight of the glycolipid). In some instances, such contaminants may be undetectable.

The glycolipid may be conjugated to an oligosaccharide, referred to herein as an oligosaccharide core unit, at one of the glucosamine residues. Such oligosaccharide may comprise 10 or fewer sugars. The sugars may be galactose, glucose and/or fucose. The oligosaccharides are typically conjugated to the diglucosamine via an acid-labile linkage such as but not limited to a ketosidic linkage.

The Bacteroides glycolipid may be provided in a number of forms. Those forms include the glycolipid form (i.e., the acyl-substituted diglucosamine) free of the oligosaccharide core unit or the tetrasaccharide unit(s) of PSA, the glycolipid conjugated to the oligosaccharide core unit, the glycolipid conjugated to the oligosaccharide core unit and to the tetrasaccharide unit(s) (i.e., lipidated PSA with varying polysaccharide lengths). The oligosaccharide core unit, by virtue of its hydrophilicity, helps to render the glycolipid more water soluble, therefore in some instances the glycolipid is conjugated to the oligosaccharide core unit. In addition, any of the foregoing forms may be provided in a cell-free form or a membrane-free form, or alternatively they may be provided bound to a membrane, such as an outer membrane, or bound to a cell such as a Bacteroides cell such as B. fragilis. If provided as a Bacteroides cell such as B. fragilis, such cells may be inactivated or attenuated. Alternatively, it may be live and unattenuated (such as in a live active culture), and in this form in some instances may be provided in a foodstuff such as a probiotic (e.g., in a yogurt, supplement, etc.).

The glycolipid may be obtained from Bacteroides spp., including but not limited to B. fragilis. One source, for example, may be a mutant non-naturally occurring strain of B. fragilis that overexpresses PSA relative to other polysaccharides. Another source may be a mutant non-naturally occurring strain of B. fragilis that expresses only PSA.

The Bacteroides glycolipid may be extracted from Bacteroides spp. using a phenol/water extraction, and DNase, RNase and pronase treatments. The subsequent extraction steps influence whether the glycolipid is harvested in a conjugated or an unconjugated form. For example, if the material is then treated with deoxycholate (DOC), the polysaccharide moiety (i.e., the repeating tetrasaccharide moiety) of PSA is removed, and the resultant material is the glycolipid conjugated to the oligosaccharide core unit. The oligosaccharide core unit, by virtue of its hydrophilicity, helps to render the lipid component more water soluble. If the material is treated instead or in addition with acid hydrolysis, the acid-labile linkage between the oligosaccharide core unit and the acyl-substituted diglucosamine is cleaved and the acyl-substituted diglucosamine alone is obtained.

Polysaccharide A

The polysaccharide component of lipidated PSA, referred to herein as PSA, comprises a tetrasaccharide repeating unit shown below. It possesses zwitterionic behavior as conferred by a positive charge on its free amine group and a negative charge on its free carboxyl group (per repeating tetrasaccharide unit). Its naturally occurring state has been reported to comprise over 60 tetrasaccharide repeating units (e.g., up to and including in some instances about 100, or about 200, or about 300 repeated units on average), and it has an average molecular size of about 150 kD (with a range of about 75 kD to 240 kD).

The repeating tetrasaccharide unit of PSA has a structure as follows:

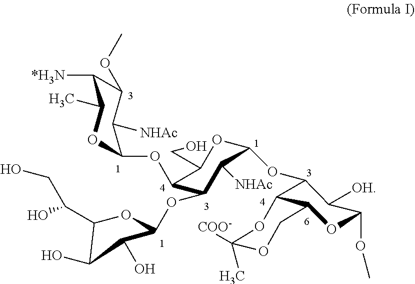

The tetrasaccharide repeating unit may also be expressed as follows:

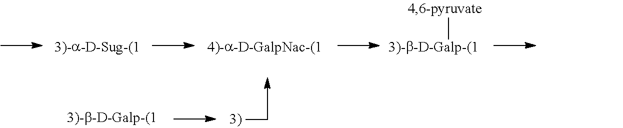

Synthetic forms of lipidated PSA may comprise comprising various ranges of tetrasaccharide units (e.g., 1-60, 1-50, 1-40, 1-30, 1-20, 1-10, 1-9, 1-8, 1-7, 1-6, or 1-5 tetrasaccharide units). Such shorter variants can be obtained by depolymerizing naturally occurring lipidated PSA or by depolymerizing PSA obtained from lipidated PSA. PSA can be depolymerized using for example chemical means (e.g., using reactive oxygen species or reactive nitrogen species such as but not limited to nitrogen monoxide, as described in Duan and Kasper, Glycobiology, 2011, 21(4):401-409), mechanical means, and/or enzymatic means that are known in the art.

Also contemplated are synthetic forms of lipidated PSA comprising more than 300 repeating tetrasaccharide units, including without limitation 350, 400, 500, 600, 700, 800, 900 or 1000 units or more.

The polysaccharide component may be covalently conjugated to the glycolipid, or in certain synthetic forms it may be unconjugated to the glycolipid. If covalently conjugated, it may be conjugated via a glycosidic bond to the oligosaccharide core unit. In other embodiments, it may be conjugated via a ketosidic bond or other acid labile bond or via a bond such as an ester, an amide, or an ether bond to form a non-naturally occurring lipidated PSA.

The glycolipids may also be provided in the form of outer membrane vesicles (OMV). In this form, the glycolipids may be provided as lipidated PSA that is anchored in the OMV or they may be provided anchored in the OMV in another manner. Methods for making OMV from bacterial species, including Bacteroides spp., are provided in US Published Applications 2013/0121966, 2015/0017664, and 2017/0145061.

Further, the glycolipids may be provided in the form of intact bacterial cells, and such cells may be live, active cells (as in a live active bacterial culture), or they may be inactivated and/or attenuated.

### Viral Infections

The glycolipids and compositions thereof may be administered to a subject having or at risk of having a viral infection. The glycolipids and compositions thereof may be used and/or administered with another therapeutic such as an anti-viral agent. Anti-viral agents include immunoglobulin, amantadine, nucleoside analogue, nonnucleoside analogue, biflavanoid and protease inhibitor. In one embodiment, the protease inhibitor is indinavir, saquinavir, ritonavir, and nelfinavir. In another embodiment, the biflavanoid is robustaflavone, amentoflavone, or a derivative or salt thereof. In yet another embodiment, the non-nucleoside analogue is selected from the group consisting of delavirdine, nevirapine, efavirenz, alpha-interferon, recombinant CD4, amantadine, rimantadine, ribavirin and vidarabine. The anti-viral may be remdesivir.

The virus may be a picornavirus (e.g., polio virus, foot and mouth disease virus), calicivirus (e.g., norovirus), togavirus (e.g., sindbis virus, the equine encephalitis viruses, chikungunya virus, rubella virus, Ross River virus, bovine diarrhea virus, hog cholera virus), flavivirus (e.g., dengue virus, West Nile virus, yellow fever virus, Japanese encephalitis virus, St. Louis encephalitis virus, tick-borne encephalitis virus), coronavirus (e.g., SARS, MERS, SARS-CoV-2, SARS-CoV-1, human coronaviruses (common cold), swine gastroenteritis virus), rhabdovirus (e.g., rabies virus, vesicular stomatitis viruses), filovirus (e.g., Marburg virus, Ebola virus), paramyxovirus (e.g., measles virus, canine distemper virus, mumps virus, parainfluenza viruses, respiratory syncytial virus, Newcastle disease virus, rinderpest virus), orthomyxovirus (e.g., human influenza viruses, avian influenza viruses, equine influenza viruses), bunyavirus (e.g., hantavirus, LaCrosse virus, Rift Valley fever virus), arenavirus (e.g., Lassa virus, Junin virus (JUNV), Machupo virus), reovirus (e.g., human reoviruses, human rotavirus), birnavirus (e.g., infectious bursal virus, fish pancreatic necrosis virus), retrovirus (e.g., HIV-1, HIV-2, HTLV-1, HTLV-2, bovine leukemia virus, feline immunodeficiency virus, feline sarcoma virus, mouse mammary tumor virus), hepadnavirus (e.g., hepatitis B virus), parvovirus (e.g., human parvovirus B, canine parvovirus, feline panleukopenia virus), papovavirus (e.g., human papillomaviruses (HPV), SV40, bovine papillomaviruses), adenovirus (e.g., human adenovirus, canine adenovirus, bovine adenovirus, porcine adenovirus), herpes virus (e.g., herpes simplex viruses such as HSV-1 and HSV-2, varicella-zoster virus, infectious bovine rhinotracheitis virus, human cytomegalovirus, human herpesvirus 6), poxvirus (e.g., vaccinia, fowlpoxviruses, raccoon poxvirus, skunkpox virus, monkeypoxvirus, cowpox virus, musculum contagiosum virus), poliovirus or rhinovirus.

The virus may be severe acute respiratory syndrome (SARS) causing virus, Middle East respiratory syndrome (MERS) causing virus, severe acute respiratory syndrome coronavirus 2 (also known as SARS-CoV-2 and 2019-nCoV, cause of COVID-19), or, SARS-CoV-1.

The virus may be hepatitis A virus (HAV), hepatitis C virus (HCV), Epstein Barr Virus (EBV), or Rous Sarcoma Virus (RSV), Enterovirus 71 (EV71), Coxsackieviruses, including serotypes A1, A4, A6, A10, and A16, Human enterovirus A (HEA), pestiviruses, measles, smallpox, cowpox, Vesicular Stomatitis Virus (VSV), Zika virus, HIV, Herpes simplex virus 1, Herpes simplex virus 2, cytomegalovirus, hepatitis A virus, hepatitis B virus, hepatitis C virus, human papilloma virus, rotavirus, adenovirus, influenza A virus, respiratory syncytial virus, varicella-zoster virus, small pox, monkey pox.

In some embodiments, the virus is a coronavirus, flavivirus, hepadnavirus, herpesvirus, orothomyxovirus, filovirus, arenavirus, calcivirus, togavirus or paramyxoviruses.

In some embodiments, the virus is SARS coronavirus, influenza virus, Ebola virus, norovirus, Zika virus, West Nile virus, Dengue virus, herpesvirus, equine encephalitis virus, respiratory syncytial virus (RSV), cytomegalovirus, rabies virus, or measles virus.

The subject may be administered the glycolipid repeatedly during a period of time or situation in which the subject is likely to be exposed to a virus. For example, the glycolipid may be administered daily, every 2, 3, 4, 5, 6 day, weekly, every 2, 3 weeks or monthly. If the subject has been exposed to a virus, or a subject who has been exposed to the virus, then the subject may be treated immediately and regularly after such exposure for a period of time. The period of time may be the incubation period of the virus (i.e., the average time it takes for symptoms to appear after exposure). The subject may be treated ahead of contact with another who is carrying the virus or has likely been exposed to the virus, and such treatment may continue beyond such contact, including for the length of the incubation period to ensure the subject manifests no symptoms associated with an infection by the virus (e.g., a fever and/or chills, muscle aches, coughing, respiratory difficulty, aberrant cardiovascular manifestations such as aberrant clotting, headaches, rashes, etc.).

The subject to be treated may therefore manifest no symptoms ahead of treatment. The subject may be tested for the presence of the virus or antibodies to the virus following treatment and/or following the incubation period. A subject who has been treated may be negative for the virus even though the subject has been in contact with the virus. A subject who has been treated may be positive for the virus but may have experienced no or diminished symptoms associated with the virus, particularly during the incubation period. A subject who has been treated may be positive for anti-viral antibodies but may have experienced no or diminished symptoms associated with the virus, particularly during the incubation period. Diminished symptoms include symptoms of lesser severity and/or of shorter duration than those experienced by untreated and infected subjects.

In some instances, the glycolipid is administered in amounts effective to induce IFNbeta in the subject at a level and for a time considered to be protective. Thus, the subject may be administered the glycolipid throughout a time during which the subject is more likely to be exposed to the virus, and the glycolipid is administered in an amount to induce sufficiently protective levels of IFNbeta. IFNbeta levels may be measured from blood samples of the subject.

In some embodiments, the subject to be treated is at a risk of exposure to the virus that is higher than the risk of exposure of the general public. Such subjects may include those medical professionals, hospital workers, and those who are hospitalized.

In some embodiments, the subject to be treated is at risk of exposure to one more than one virus, and optionally the risk is higher than the risk of exposure to the general public. Such subjects may include those medical professionals, hospital workers, and those who are hospitalized.

In some embodiments, the subject to be treated is at risk of developing an infection upon exposure to a virus, and optionally the risk is higher than the risk of developing an infection by the general public. Such subjects may include those who were, are and/or will be treated with one or more antibiotics and/or with a broad spectrum antibiotic. Such subjects may include those who have received another therapy that has caused imbalance in their gut microbiome, such as but not limited to a cancer chemotherapy.

### TLR4-Dependent Conditions

This disclosure further provides methods for treating TLR4-dependent conditions. TLR4-dependent conditions are conditions that are mediated at least in part by TLR4 signaling. Such signaling may be aberrant or uncontrolled or may lead to downstream events that are detrimental to the subject. Examples of TLR4-dependent conditions include bacterial infections, sepsis, organ fibrosis, certain inflammatory conditions, and others known in the art. In some instances, the TLR4-dependent condition is not sepsis. In some instances, the subject does not have sepsis. In some instances, the TLR4-dependent condition is not an autoimmune disorder. In some instances, the subject does not have an autoimmune disorder.

The glycolipids of this disclosure may be used in such subjects to modulate TLR4 signaling in quality and/or quantity. The glycolipids may be used to engage TLR4, thereby competing with the condition-causing agent for binding and signal induction through TLR4, with the glycolipids inducing a different downstream response than the one induced by the condition-causing agent. In such situations, the glycolipids may be used to reduce or eliminate the side effects of the condition-causing agent. The glycolipids may be administered once or repeatedly while the condition is manifested. The administration regimen of the glycolipids may also depend on the nature of the condition and the condition-causing agent. If the condition-causing agent is a TLR4 agonist or antagonist that is deliberately administered to the subject, then the glycolipid may be administered alongside of or at a spaced time interval from the TLR4 agonist or antagonist.

In related aspects, the glycolipids may be used to modulate TLR4 signaling. The glycolipids may be used to reduce signaling through TLR4 in subjects who are experiencing elevated TLR4 signaling, such as may be the case if the subject is being administered a TLR4 agonist. The glycolipids may be used to increase signaling through TLR4 in subjects who are experiencing reduced TLR4 signaling, such as may be the case if the subject is being administered a TLR4 agonist.

The TLR4 agonist or antagonist may be inducing signal through the MyD88-dependent pathway following TLR4 engagement. The glycolipids of this disclosure may cause a shift towards the TRIF pathway, thereby reducing signaling through MyD88 and its aberrant effects.

The subject may be one who has been administered, or is being administered, or will be administered a TLR4 agonist or antagonist. TLR4 agonists and TLR4 antagonists may be compounds that are prescribed and intended to treat another condition but which adversely impact TLR4 signaling through TLR4 engagement. Examples of TLR4 agonists are buprenorphine, carbamazepine, fentanyl, levorphanol, lipopolysaccharide (LPS), methadone, morphine, oxycarbezepine, oxycodone, pethidine, tapentadol and morphine-3-glucuronide. Examples of TLR4 antagonists are naloxone, naltrexone, propentofylline, palmitoylethanolamide, amitriptyline, cyclobenzaprine, ketotifen, imipramine, mianserin, ibudilast, pinocembrin and resatorvid.

In still other instances, the glycolipid may be used to stimulate or enhance a TLR4-mediated immune response. This may benefit subjects who fail to induce such immune responses in the absence of the glycolipid, or who need a boost in order to do so. The glycolipid may be used, in any one or combination of its forms, as a vaccine adjuvant. Additionally, the glycolipid may be used to induce immunosuppressive or immunoregulatory activity via TLR4-mediated signaling. Further data (not shown) evidence the ability of the glycolipid to induce a variety of these downstream effects, including for example regulating IL-10 secretion by T cells through TLR4/IFNb through induction of a tolerogenic DC phenotype and type I regulatory T (Tr1) cells. Tr1 cells regulate tolerance towards antigens of any origin. Tr1 cells are self or non-self antigen specific and they play a role in inducing and maintaining peripheral tolerance and suppressing tissue inflammation in autoimmunity and graft vs. host disease. It was found that PSA signals through IFNβ to induce an immunoregulatory phenotype in antigen presenting cells (APCs) and enhance IL-10 secretion by CD4+ T cells. Importantly, the protective effects of PSA in a mouse autoimmune disease model were dependent on IFN-I signaling, demonstrating the important tolerogenic role of commensal-induced IFNβ. These activities indicate that the glycolipids can be used for a variety of applications, prophylactically or therapeutically.

### Subjects

The methods provided herein are to use in a subject. As used herein, a “subject” may be a human or a non-human subject. Non-human subjects include, for example, agricultural animals such as cows, pigs, horses, goats, sheep, bison, etc., and domesticated animals such as dogs and cats, or other domesticated animals. In certain embodiments, the subject is human.

In some embodiments, the subject is one who has been administered, and/or is being administered, and/or will be administered a broad-spectrum antibiotic or other treatment that will create an imbalance in the gut microbiome. A broad spectrum antibiotic as used herein in an antibiotic that acts against more than one bacterial species. Some broad spectrum antibiotics act on a variety of bacterial species including both gram-positive and gram-negative species. Examples include aminoglycosides (except for streptomycin), ampicillin, amoxicillin/clavulanic acid (Augmentin), carbapenems (e.g. imipenem), piperacillin/tazobactam. quinolones (e.g. ciprofloxacin), tetracyclines, chloramphenicol, ticarcillin, and trimethoprim/sulfamethoxazole (Bactrim).

### Treatment

The terms “treat,” “treating” and “treatment,” as used herein, means reducing the frequency or severity with which symptoms of a disease or condition are experienced by a subject by virtue of administering an agent or compound of this disclosure to the subject.

The glycolipids of this disclosure may be used to reduce onset of a viral infection and optionally a condition caused by the viral infection. The glycolipids and compositions thereof may be capable of reducing or preventing onset of a viral infection or side effect of exposure caused by one or more viruses when administered to a subject.

The glycolipids of this disclosure may be formulated as pharmaceutical compositions that include a pharmaceutically acceptable excipient. The term “pharmaceutically acceptable carrier” as used herein is intended to include diluents such as saline and aqueous buffer solutions.

### Administration Routes

The glycolipids and compositions (e.g. pharmaceutical compositions) thereof can be administered to a subject by any known manner, for example, subcutaneous, intramuscular, intravenous, intradermal, by oral administration, inhalation, transdermal application, intravaginal application, topical application, intranasal or rectal administration. In one embodiment, the compound may be administered intranasally, such as inhalation.

In certain embodiments, compositions disclosed herein are administered orally or into the gastrointestinal tract for example using enteric-coated pills, capsules or tablets. The compositions, including the glycolipids in conjugated or unconjugated form, in lipidated PSA form, in the form of Bacteroides outer membrane, or in inactivated or attenuated Bacteroides cells, may be formulated as food, including yogurts, nutritional bars, and the like.

### Kits

The glycolipids and/or compositions thereof may be provided in a kit for in vitro and/or in vivo use. The kits may include a suitable container, the glycolipid composition, and one or more additional agents such as other anti-viral agents. The compositions may be partially or wholly dehydrated and/or in a ready to use form. Kits may be stored at room temperatures or at refrigerated temperatures depending on the particular formulation.

The following Examples are included for purposes of illustration and are not intended to limit the scope of the invention.

### Examples

### Introduction

The type I interferon (IFN-I) response is a crucial mediator of antiviral immunity and homeostatic immune system regulation. However, the source of IFN-I signaling under homeostatic conditions remains unclear. In this study, we discovered that the commensal microbiota regulates the IFN-I response both locally and systemically through induction of IFNβ by colon lamina propria dendritic cells. Moreover, the molecular mechanism by which a specific commensal microbe induces IFNβ was identified. Indeed, we found that outer membrane-associated glycolipids of human gut commensal microbes belonging to the Bacteroidetes phylum induce expression of IFNβ. Using Bacteroides fragilis, as well as its outer membrane associated polysaccharide A as the paradigm, we determined that IFNβ expression was induced via the TLR4-TRIF signaling pathway. Antiviral activity of this purified microbial molecule against infection with either vesicular stomatitis virus (VSV) or influenza was demonstrated to be dependent on the induction of IFNβ. In an in vivo model of VSV infection, we discovered that commensal-induced IFNβ modulates the health of the host by regulating natural resistance to virus infection. Due to the importance of IFN-Is in antiviral immunity and other vital aspects of human physiology, discovery of an IFNβ-inducing microbial molecule represents a potential novel therapeutic approach for the treatment of a variety of human diseases.

One mechanism by which microbes can modulate the host immune system is through regulation of cytokine signaling. Type I interferons (IFN-Is) are a family of structurally similar cytokines, consisting primarily of two different classes of proteins, interferon-α (IFNα) and interferon-β(IFN-β), all of which signal through the common type I interferon receptor (IFNAR). (Theofilopoulos et al., 2005) IFN-I expression is regulated at the transcriptional level, and can be induced at high levels in all nucleated cells in response to sensing of pathogens by pattern recognition receptors (PRRs). (Ivashkiv and Donlin, 2014) Indeed, IFN-Is play a critical role in the response to the majority of virus infections, through induction of a restrictive anti-viral state in cells, induction of apoptosis in virus-infected cells, and through regulation of immune cell subsets crucial to the antiviral response. (McNab et al., 2015; Muller et al., 1994; Stetson and Medzhitov, 2006; Yan and Chen, 2012) Several recent studies have demonstrated that IFN-Is are not just present during infection, but are expressed constitutively at low-levels and possess vital homeostatic functions. (Gough et al., 2012; Kole et al., 2013) Indeed, IFN-Is have the capacity to regulate the development and/or activation of virtually every immune effector cell, playing important roles in the anti-inflammatory and anti-tumor responses. (Gough et al., 2012) Despite this importance, the mechanism by which IFN-Is are induced and regulated in the absence of infection has yet to be elucidated. Indeed, research on microbial regulation of the IFN-I response has been largely limited to diseased states or administration of exogenous, and often synthetic, microbial ligands.

Due to the importance of IFN-Is to the health of the organism, both in defense against pathogens and maintenance of homeostasis, it is of critical importance to gain a mechanistic understanding of how the constitutive IFN-I response is regulated in healthy individuals and might be disrupted leading to disease, with the potential to lead to new therapeutic strategies. We hypothesized that the commensal microbiota supplies PRR signaling to regulate the IFN-I response in the absence of infection. In this study, we discovered that the commensal microbiota regulates the IFN-I response both locally and systemically through induction of IFNβ by colonic lamina propria (LP) dendritic cells (DCs), enhancing resistance to virus infection. Moreover, the molecular mechanism by which a specific commensal microbe induces IFNβ was identified. Indeed, we found that outer membrane-associated glycolipids of human gut commensal microbes belonging to the Bacteroidetes phylum induce expression of IFNβ. Using Bacteroides fragilis (B. fragilis) as the paradigm, we determined that signaling to initiate IFNβ expression was through the toll-like receptor 4-(TLR4) TIR-domain-containing adapter-inducing interferon-β (TRIF) pathway.

### Results

### The Commensal Microbiota Regulates Host ISG Expression Through IFNβ

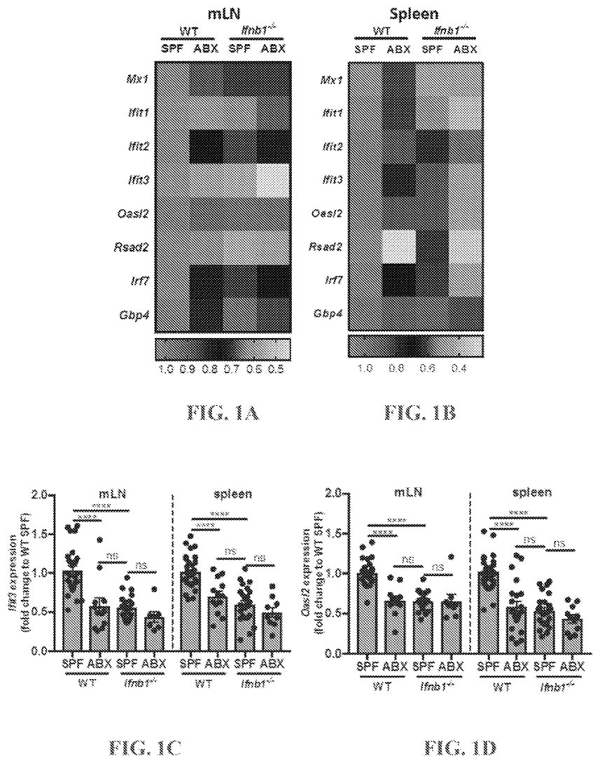

To investigate the role of the commensal microbiota in the host IFN-I response, the effect of microbiota depletion on downstream interferon stimulated gene (ISG) expression in various murine tissues was analyzed. The vast majority of commensal microbes reside in the GI tract. (Savage, 1977; Turnbaugh et al., 2007) Analysis was therefore performed to determine effects of the microbiota on ISG expression in the intestinal immune compartment, in the mesenteric lymph nodes (mLN), and systemically, in the spleen. Age- and gender-matched wild type (WT) specific pathogen free (SPF) C57BL/6 mice were administered either vehicle control or a cocktail of broad-spectrum antibiotics (ABX) in the drinking water for 7 days to deplete the microbiota. The relative expression levels of a panel of ISGs were analyzed by quantitative reverse transcription polymerase chain reaction (qRT-PCR). A reduction in ISG expression was observed in both the mLN and spleen of WT ABX mice compared to WT SPF mice (FIGS. 1A-1D, Tables 1-2), demonstrating a role for microbiota regulation of the IFN-I response both locally and systemically.

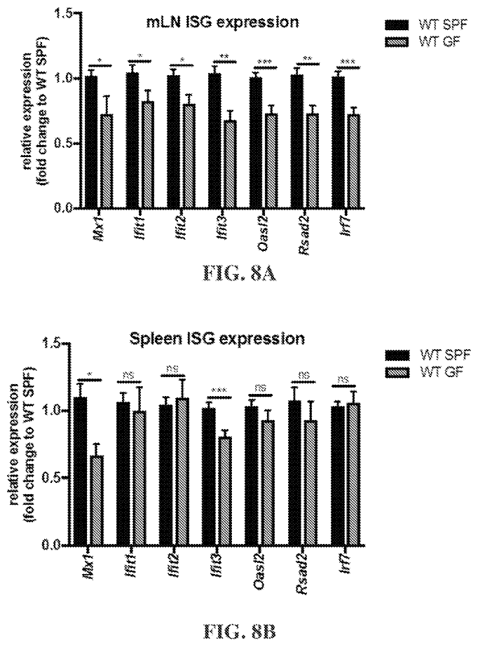

Antibiotics can have off-target effects, acting in some instances directly on the host rather than on the commensal microbes themselves. (Morgun et al., 2015) A second method of microbiota depletion was therefore used to confirm a direct role of the commensal microbiota on the observed phenotype. Splenic and mLN ISG expression was analyzed in WT C57BL/6 germ free (GF) mice devoid of all microbes compared to WT C57BL/6 SPF mice. Consistent with the antibiotics-treated mice, a reduction in mLN and splenic ISG expression was observed (FIGS. 8A-8B, Tables 1-2).

Of the mammalian IFN-Is, IFNβ is unique with respect to its homeostatic functions and expression pattern. (Schreiber and Piehler, 2015) It was therefore hypothesized that the commensal microbiota induces the constitutive IFN-I response, as evidenced by ISG expression, specifically through regulation of IFNβ. ISG expression levels were compared by qRT-PCR between WT and IFNβ deficient (Ifnb1^{-/-}) SPF C57BL/6 mice. A significant reduction in ISG expression was observed in the mLN and spleen of Ifnb1^{-/-} mice compared to WT mice (FIGS. 1A-1D, Tables 1-2), confirming a role for IFNβ in the homeostatic IFN-I response. Interestingly, the magnitude of reduction was comparable to that observed upon microbiota depletion, with no significant difference between WT ABX and Ifnb1^{-/-} SPF expression levels. To further test whether commensal-induced IFNβ is required for ISG expression, the microbiota of Ifnb1^{-/-} mice was depleted by antibiotics administration. Antibiotics treatment had no additional effect on mLN or splenic ISG expression in Ifnb1^{-/-} mice (FIGS. 1A-1D, Tables 1-2), demonstrating that commensal microbes regulate the IFN-I response specifically through IFNβ.

TABLE 1
Statistical analysis of mLN ISG expression. mLNs were harvested from age- and
gender-matched C57BL/6 WT GF mice and from WT or Ifnb1^{-/-} SPF mice with and without
broad spectrum antibiotics (ABX) treatment. RNA was isolated from whole tissue samples and
qRT-PCR was performed to analyze ISG expression levels. Fold change gene expression was
calculated compared to WT SPF mice using the delta-delta CT method, with ActB as the
reference gene.
mLN fold difference gene expression to WT SPF

| | | | | WT GF | | | | | WT ABX | | | | |
|---|---|---|---|---|---|---|---|---|---|---|---|---|---|
| | WT SPF | | | | | | p value to | p value to | | | | p value to | p value to |
| gene | Mean | SEM | N | Mean | SEM | N | WT SPF | Ifnb1^{-/-} SPF | Mean | SEM | N | WT SPF | Ifnb1^{-/-} SPF |
| Mx1 | 1.02 | 0.04 | 25 | 0.73 | 0.14 | 7 | 0.4373 (ns) | 0.9975 (ns) | 0.68 | 0.07 | 13 | 0.0073** | 0.7648 (ns) |
| Ifit1 | 1.04 | 0.06 | 27 | 0.83 | 0.08 | 11 | 0.1742 (ns) | 0.3093 (ns) | 0.61 | 0.06 | 12 | 0.0002*** | 0.9997 (ns) |
| Ifit2 | 1.03 | 0.04 | 27 | 0.81 | 0.07 | 11 | 0.275 (ns) | 0.8272 (ns) | 0.75 | 0.11 | 12 | 0.0797 (ns) | 0.9791 (ns) |
| Ifit3 | 1.04 | 0.06 | 27 | 0.68 | 0.07 | 11 | 0.0019** | 0.6811 (ns) | 0.58 | 0.10 | 12 | <0.0001**** | 0.9986 (ns) |
| Oas/2 | 1.01 | 0.03 | 26 | 0.73 | 0.06 | 11 | 0.0002*** | 0.7673 (ns) | 0.66 | 0.06 | 11 | <0.0001**** | >0.9999 (ns) |
| Rsad2 | 1.03 | 0.05 | 25 | 0.74 | 0.06 | 11 | 0.0008*** | 0.1064 (ns) | 0.63 | 0.06 | 10 | <0.0001**** | 0.8457 (ns) |
| Irf7 | 1.02 | 0.03 | 27 | 0.73 | 0.05 | 11 | <0.0001**** | 0.9793 (ns) | 0.77 | 0.06 | 12 | 0.0009*** | 0.6858 (ns) |
| Gbp4 | 1.01 | 0.03 | 28 | 0.78 | 0.06 | 14 | <0.0001**** | 0.0029** | 0.81 | 0.07 | 12 | 0.0197* | 0.3071 (ns) |

| | Ifnb1^{-/-} SPF | | | | | Ifnb1^{-/-} ABX | | | | |
|---|---|---|---|---|---|---|---|---|---|---|
| | | | | p value to | | | | | p value to | p value to |
| gene | Mean | SEM | N | WT SPF | N | Mean | SEM | N | WT SPF | Ifnb1^{-/-} SPF |
| Mx1 | 0.83 | 0.06 | 17 | 0.1552 (ns) | | 0.69 | 0.09 | 8 | 0.0419* | 0.8966 (ns) |
| Ifit1 | 0.63 | 0.05 | 21 | <0.0001**** | 21 | 0.67 | 0.09 | 7 | 0.0138* | 0.9979 (ns) |
| Ifit2 | 0.68 | 0.04 | 21 | 0.0025** | 21 | 0.72 | 0.22 | 8 | 0.1184 (ns) | 0.9978 (ns) |
| Ifit3 | 0.56 | 0.04 | 21 | <0.0001**** | 21 | 0.43 | 0.07 | 7 | <0.0001**** | 0.8118 (ns) |
| Oas/2 | 0.66 | 0.03 | 21 | <0.0001**** | 21 | 0.66 | 0.08 | 8 | <0.0001**** | >0.9999 (ns) |
| Rsad2 | 0.56 | 0.03 | 20 | <0.0001**** | 20 | 0.56 | 0.05 | 7 | <0.0001**** | >0.9999 (ns) |
| Irf7 | 0.69 | 0.03 | 21 | <0.0001**** | 21 | 0.75 | 0.05 | 8 | 0.0024** | 0.901 (ns) |
| Gbp4 | 0.96 | 0.05 | 19 | 0.6544 (ns) | 19 | 0.85 | 0.06 | 8 | 0.1917 (ns) | 0.7471 (ns) |

N = number of samples analyzed,
SEM = standard error of the mean,
ns = not significant,
*p < 0.05,
** p < 0.01,
*** p < 0.001,
****p < 0.0001.

TABLE 2
Statistical analysis of spleen ISG expression. Spleens were harvested from age- and
gender-matched C57BL/6 WT GF mice and from WT or Ifnb1^{-/-} SPF mice with and without
broad spectrum antibiotics (ABX) treatment. RNA was isolated from whole tissue samples and
qRT-PCR was performed to analyze ISG expression levels. Fold change gene expression was
calculated compared to WT SPF mice using the delta-delta CT method, with ActB as the
reference gene.
Spleen fold difference gene expression to WT SPF

| | |
|---|---|
| WT GF | WT ABX |

| | WT SPF | | | | | | p value to | p value to | | | | p value to | p value to |
|---|---|---|---|---|---|---|---|---|---|---|---|---|---|
| gene | Mean | SEM | N | Mean | SEM | N | WT SPF | Ifnb1^{-/-} SPF | Mean | SEM | N | WT SPF | Ifnb1^{-/-} SPF |
| Mx1 | 1.10 | 0.09 | 38 | 0.67 | 0.08 | 21 | 0.0019** | 0.5028 (ns) | 0.83 | 0.07 | 15 | 0.2171 (ns) | 0.0658 (ns) |
| Ifit1 | 1.07 | 0.07 | 29 | 1.00 | 0.17 | 8 | 0.9832 (ns) | 0.0023** | 0.61 | 0.05 | 20 | <0.0001**** | 0.871 (ns) |
| Ifit2 | 1.04 | 0.06 | 31 | 1.10 | 0.14 | 16 | 0.9892 (ns) | 0.002** | 0.87 | 0.07 | 18 | 0.5135 (ns) | 0.2127 (ns) |
| Ifit3 | 1.02 | 0.04 | 41 | 0.81 | 0.05 | 24 | 0.003** | 0.0048** | 0.75 | 0.05 | 21 | 0.0001*** | 0.5977 (ns) |
| Oas/2 | 1.03 | 0.04 | 38 | 0.93 | 0.07 | 21 | 0.6461 (ns) | 0.0005*** | 0.59 | 0.07 | 21 | <0.0001**** | >0.9999 (ns) |
| Rsad2 | 1.08 | 0.09 | 27 | 0.93 | 0.13 | 13 | 0.8672 (ns) | 0.2781 (ns) | 0.25 | 0.04 | 13 | <0.0001**** | 0.1149 (ns) |
| Irf7 | 1.03 | 0.04 | 38 | 1.06 | 0.08 | 2 | 0.9911 (ns) | <0.0001**** | 0.69 | 0.03 | 21 | <0.0001**** | 0.83 (ns) |
| Gbp4 | 1.02 | 0.04 | 27 | 0.65 | 0.05 | 15 | <0.0001**** | 0.0101* | 0.89 | 0.04 | 21 | 0.2753 (ns) | 0.9962 (ns) |

| | Ifnb1^{-/-} SPF | | | | | Ifnb1^{-/-} ABX | | | | |
|---|---|---|---|---|---|---|---|---|---|---|
| | | | | p value to | | | | | p value to | p value to |
| gene | Mean | SEM | N | WT SPF | N | Mean | SEM | N | WT SPF | Ifnb1^{-/-} SPF |
| Mx1 | 0.47 | 0.04 | 25 | <0.0001**** | 17 | 0.47 | 0.07 | 11 | 0.0002*** | >0.9999 (ns) |
| Ifit1 | 0.52 | 0.05 | 23 | <0.0001**** | 21 | 0.37 | 0.06 | 11 | <0.0001**** | 0.7177 (ns) |
| Ifit2 | 0.64 | 0.06 | 20 | 0.0012** | 21 | 0.56 | 0.13 | 8 | 0.0085** | 0.9879 (ns) |
| Ifit3 | 0.58 | 0.04 | 27 | <0.0001**** | 21 | 0.43 | 0.07 | 11 | <0.0001**** | 0.7042 (ns) |
| Oas/2 | 0.54 | 0.05 | 27 | <0.0001**** | 2 | 0.43 | 0.05 | 11 | <0.0001**** | 0.5122 (ns) |
| Rsad2 | 0.63 | 0.11 | 20 | 0.0064** | 20 | 0.31 | 0.07 | 7 | 0.0007*** | 0.4625 (ns) |
| Irf7 | 0.62 | 0.05 | 27 | <0.0001**** | 2 | 0.46 | 0.05 | 11 | <0.0001**** | 0.3749 (ns) |
| Gbp4 | 0.92 | 0.07 | 16 | 0.5894 (ns) | 19 | 0.85 | 0.07 | 11 | 0.2112 (ns) | 0.9356 (ns) |

N = number of samples analyzed,
SEM = standard error of the mean,
ns = not significant,
*p<0.05,
**p<0.01,
***p<0.001,
****p<0.0001.

Further data (not shown) support the finding that glycolipids of this disclosure modulate a cluster of genes that are functionally relevant to anti-viral immunity. Among various genes examined, it was found that activation of splenic dendritic cells (DC) by tri-acylated and tetra-acylated monophosphorylated glycolipids provided herein resulted in the induction of a cluster of genes that are found in ISG modules, including for example Oas1a/b, Oasl1/2, Ifit1/2, Mx1/2, and Stat1/2. These gene sets are all associated with transcriptomic changes in response to type I IFN in DCs and plasmacytoid DCs. These data strongly suggest that immunomodulatory changes driven by under-acylated monophosphorylated glycolipids could lead to anti-viral immune responses mediated by type I IFNs (e.g., IFN alpha and/or IFN beta) via IFNAR1 and cellular interaction of DCs with T cells.

### IFNβ is Expressed by Colonic Dendritic Cells

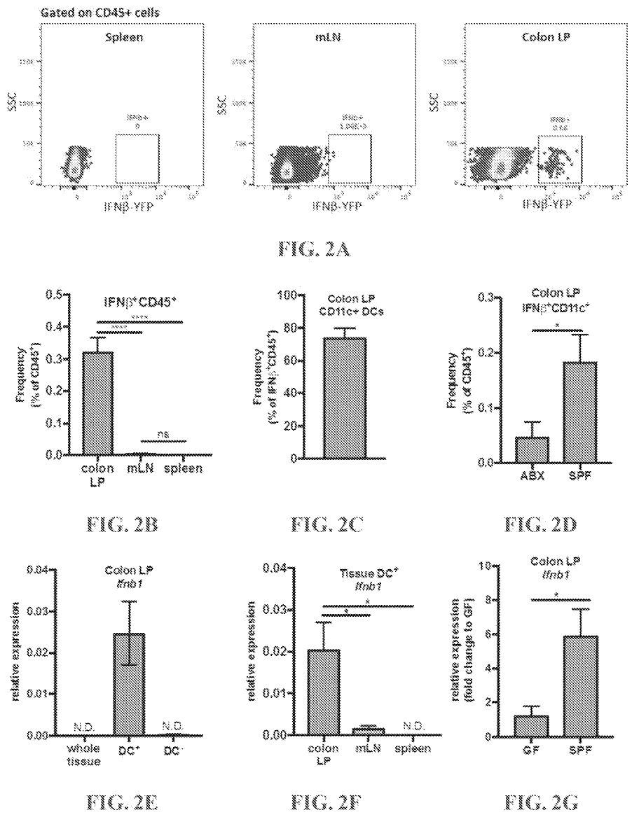

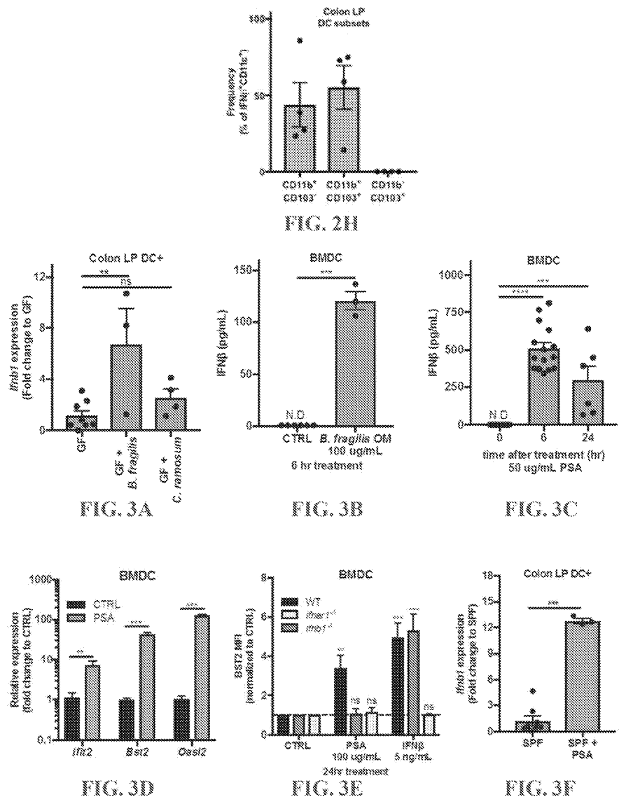

To investigate the source of commensal-induced IFNβ under homeostatic conditions, flow cytometric analysis was performed on tissues harvested from IFNβ-yellow fluorescent protein (YFP) reporter mice, in which cells activated to express IFNβ express YFP. (Scheu et al., 2008) By analyzing IFNβ-YFP expression in the colon LP, mLN, and spleen, IFNβ expression was found to be localized to the intestinal milieu, with significantly higher percentages of IFNβ-YFP⁺CD45⁺ leukocytes in the colonic LP compared to the spleen or mLN (FIGS. 2A-2B). The colon was chosen as a representative region of the GI tract because it is home to the greatest percentage of commensal organisms, both in terms of number and abundance. (Turnbaugh et al., 2007) Having identified the location of IFNβ expression, the cellular source of this cytokine was next investigated. Due to their role as sentinel cells in the immune system, dendritic cells (DCs) were hypothesized to be the main source of IFNβ under homeostatic conditions. It was found that out of the IFNβ-YFP⁺CD45⁺ cells in the colonic LP, the majority (˜74%) expressed CD11c, a molecule expressed on the surface of DCs and commonly used as a DC marker (FIG. 2C). (Singh-Jasuja et al., 2013) These findings demonstrate that colon LP dendritic cells are a major source of IFNβ under homeostatic conditions.

As in other tissues, conventional DCs (cDCs) in the colon LP represent a heterogeneous population that can be classified into two subsets based on surface marker expression. cDC1 cells are CD103+CD11b− while cDC2 cells comprise both CD103−CD11b+ and CD103+CD11b+ cells. (Stagg, 2018) To determine which DCs express IFNβ, expression of CD103 and CD11b by the IFNβ-YFP+CD11c+CD45+ colonic dendritic cells was analyzed using flow cytometry. IFNβ expression was specific to the cDC2 subset of cells, including both CD103+CD11b+ and CD103-CD11b+ cells (FIG. 2H).

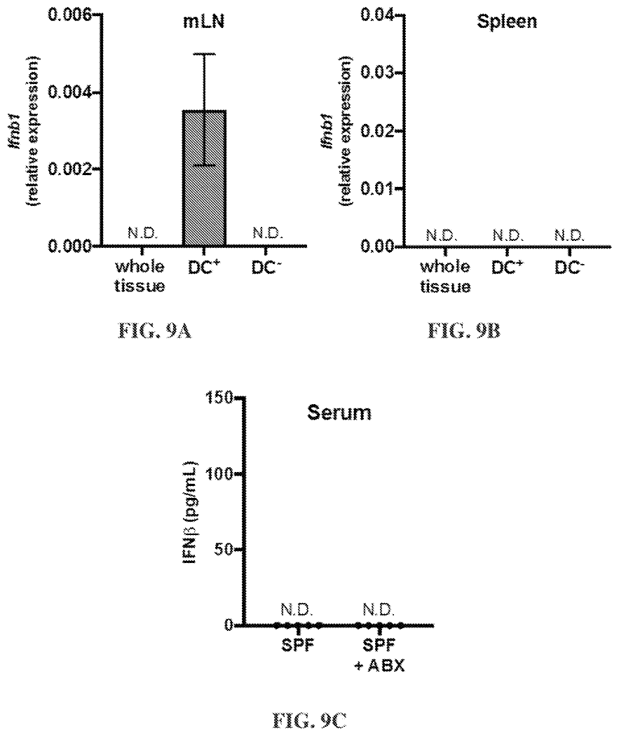

To confirm these findings, a second experimental approach was used in which DCs were isolated from the colon LP, mLN, and spleens of WT SPF mice using a magnetic bead-based cell separation method. Expression of Ifnb1 was analyzed in the whole tissue single cell suspension as well as both the DC⁺ and DC⁻ fractions of these tissues by qRT-PCR. There was no detectable Ifnb1 expression in the whole tissue or the DC⁻ fraction of the majority of colon LP samples (FIG. 2E) or in the mLN (FIG. 9A) and spleen (FIG. 9B). However, DCs in both the colon LP (FIGS. 2E-2F) and the mLN (FIG. 2F, FIG. 9A) but not the spleen (FIG. 2F, FIG. 9B) expressed Ifnb1. Interestingly, DC expression of Ifnb1 is significantly higher in the colon LP than in the mLN (FIG. 2F). These findings confirm that IFNβ is predominantly expressed by colon LP DCs.

To test the role of the microbiota in colonic DC IFNβ expression, two methods of microbiota depletion were employed. Broad-spectrum antibiotics were administered in the drinking water of IFNβ-YFP mice for 7 days, followed by flow cytometric analysis of colon LP cells. A significant reduction in the frequency of IFNβ-YFP⁺CD11c⁺ cells was observed in IFNβ-YFP mice after ABX treatment (FIG. 2D). In addition, Ifnb1 transcript levels in colon LP DCs of WT GF mice were analyzed by qRT-PCR. A significant reduction in Ifnb1 expression in WT GF mice compared to WT SPF mice was observed (FIG. 2G). Together, these results confirm that the microbiota induces IFNβ expression by colon LP DCs

Having identified effects of commensal-induced IFNβ on ISG expression not just locally, in the mLN, but also systemically in the spleen, we investigated IFNβ levels in systemic circulation, by measuring serum IFNβ levels using an enzyme-linked immunosorbent assay (ELISA). Consistent with reports that IFN-I expression is extremely low under homeostatic conditions, we were unable to detect IFNβ in the serum of WT SPF or ABX mice (FIG. 9C).

Bacteroides fragilis Induces Expression of IFNβ by Colon LP DCs

Of the trillions of commensal bacteria that inhabit the mammalian gut, one of the most prominent phyla represented is the Bacteroidetes, consisting of obligate anaerobic, Gram-negative rods, with about 25% of all colonic anaerobes belonging to the genus Bacteroides within this phylum. (Salyers, 1984; Wexler, 2007) Recently an association was reported between several species of the genus Bacteroides, pDC numbers, and a IFN-I response gene signature. (Geva-Zatorsky et al., 2017) One of the Bacteroides sp. investigated in this study was B. fragilis, a common human colonic commensal bacterial species with previously demonstrated immunomodulatory properties. (An et al., 2014; Dasgupta et al., 2014; Mazmanian et al., 2005; Mazmanian et al., 2008) We hypothesized that B. fragilis regulates colon DC IFNβ expression and thus regulates the IFN-I response. GF mice were colonized with B. fragilis strain NCTC 9343 at 4 weeks of age, followed by isolation of colon LP DCs after 2 weeks and analysis of Ifnb1 expression. As a colonization control, GF mice were colonized with a different bacterial species with demonstrated immunomodulatory properties, Clostridium ramosum (C. ramosum), thus controlling for any non-specific effects due to bacterial colonization. (Geva-Zatorsky et al., 2017; Sefik et al., 2015) We observed that colonization with B. fragilis significantly enhanced Ifnb1 expression in colon LP DCs, while C. ramosum colonization had no effect (FIG. 3A). While B. fragilis predominantly colonizes the lower intestinal tract, it is possible that it might also regulate Ifnb1 expression in other parts of the intestine. We therefore also examined Ifnb1 expression by DCs isolated from the small intestine (SI) LP of B. fragilis monocolonized mice. In contrast to the colon, colonization with B. fragilis had no observable effect on SI LP DC Ifnb1 expression (data not shown), demonstrating that regulation of DC IFNβ by this commensal organism is location specific.

B. fragilis Capsular Polysaccharide a Induces IFNβ Expression

The outer membrane (OM) of Gram-negative bacteria, such as B. fragilis, serves as the site of interaction between the bacterial cell and its environment and comprises several classes of potential immunomodulatory molecules. (Kasper and Seiler, 1975; May and Grabowicz, 2018) To test whether an OM component is responsible for IFNβ expression, an in vitro model was developed in which bone marrow-derived dendritic cells (BMDCs) isolated from WT mice were treated with OM complexes isolated from B. fragilis, followed by ELISA analysis of secreted IFNβ in the supernatants. It was found that B. fragilis OM complexes significantly induced IFNβ secretion by BMDCs (FIG. 3B). Capsular polysaccharides can be attached to the OM through covalent linkage to lipid anchors, which are inserted into the OM lipid bilayer. (Cress et al., 2014) One capsular polysaccharide of B. fragilis, polysaccharide A (PSA) has been demonstrated to have immunomodulatory properties. (Dasgupta et al., 2014; Mazmanian et al., 2005; Mazmanian et al., 2008) To test whether PSA is an IFNβ-inducing component of the B. fragilis OM, BMDCs were treated with purified PSA and secretion of IFNβ in the supernatants was analyzed by ELISA. Indeed, PSA significantly induced IFNβ secretion by BMDCs (FIG. 3C).

To confirm the ability of PSA to induce a productive IFN-I response through IFNβ, expression of downstream ISGs was analyzed by qRT-PCR in BMDCS treated for 24 hrs with PSA or vehicle control. An increase in expression of a panel of ISGs was observed in WT BMDCs treated with PSA (FIG. 3D). Expression levels of the surface-expressed ISG, bone marrow stromal cell antigen 2 (BST2), were determined by flow cytometric analysis of mean fluorescence intensity (MFI) and also found to be increased in PSA-treated compared to vehicle control WT BMDCs (FIG. 3E). Surface BST2 levels after PSA treatment were significantly reduced in IFNAR1 deficient (Ifnar1^{-/-}) and Ifnb1^{-/-} BMDCs, confirming that signaling of IFNβ through IFNAR1 is necessary for the observed increase in levels of a representative ISG by PSA. Together, these data demonstrate that PSA induces secretion of IFNβ and downstream ISG expression in vitro by BMDCs.

To confirm the relevance of these findings in vivo, Ifnb1 expression by colon LP DCs was analyzed in WT SPF mice administered 150 ug of PSA by oral gavage. A significant increase in Ifnb1 expression was observed 1.5 hrs after treatment (FIG. 3F), suggesting that PSA is capable of signaling to colon LP DCs in vivo to induce Ifnb1 expression.

B. fragilis Lipooligosaccharide Signals Through TLR4 to Induce IFNβ

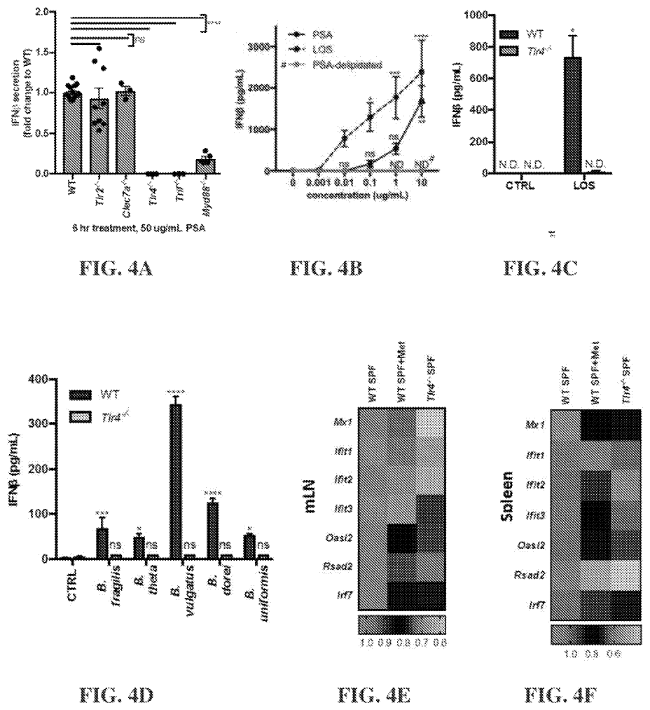

PSA has been previously demonstrated to signal via TLR2/1 heterodimers and dectin-1 on dendritic cells to induce secretion of IL-10 by CD4⁺ T cells. (Dasgupta et al., 2014; Erturk-Hasdemir et al., 2019; Round and Mazmanian, 2010) The role of TLR2 and dectin-1 in PSA induction of IFNβ was therefore investigated by comparing the response of WT, dectin-1 (Clec7c^{-/-}), and TLR2 (Tlr2^{-/-}) deficient BMDCs to PSA using ELISA to detect IFNβ secreted in the supernatants. Much to our surprise, loss of dectin-1 or TLR2 had no effect on IFNβ secretion (FIG. 4A), suggesting that PSA signals through a different and previously unidentified pathway to regulate IFNβ expression.

The majority of IFN-I inducing PRRs sense viral nucleic acid, with the bacteria-sensing TLR2, dectin-1, and TLR4 pathways representing some of the few exceptions. TLR4 senses microbial glycolipids and of the IFN-Is predominantly induces IFNβ, consistent with the signaling observed by PSA. (Lu et al., 2008; Sheikh et al., 2014) TLR4 deficient (Tlr4^{-/-}) BMDCs completely lost the ability to secrete IFNβ in response to PSA treatment (FIG. 4A), confirming that PSA signals through TLR4 to induce IFNβ secretion.

TLR4 signaling proceeds through two different signaling adapter molecules, myeloid differentiation primary response protein (MyD88) and TRIF, which activate divergent pathways to mediate downstream gene expression changes. MyD88 canonically activates the transcription factor nuclear factor kappa-light-chain-enhancer of activated B cells (NFκB) to induce proinflammatory gene expression while TRIF is reported to activate interferon regulatory factor 3 (IRF3) to mediate IFNβ expression. (Maeshima and Fernandez, 2013) Consistent with these well-described signaling events, PSA induction of IFNβ was entirely dependent on TRIF, as evidenced by the lack of increased IFNβ secretion in TRIF deficient (Trif^{-/-}) BMDCs in response to PSA (FIG. 4A). A significant decrease in IFNβ secretion was also observed in PSA-treated MyD88 deficient (Myd88^{-/-}) BMDCs (FIG. 4A), potentially due to regulatory effects that MyD88 expression has on BMDC function rather than direct induction of IFNβ by MyD88.

The canonical TLR4 ligand is LPS, the primary component of the OMs of Gram-negative bacteria. (Maeshima and Fernandez, 2013) In contrast to the archetypal Gram-negative bacterial species Escherichia coli (E. coli), the OM of B. fragilis lacks LPS but instead consists of a lipooligosaccharide (LOS), comprising a lipid A domain linked to a short oligosaccharide. (Lindberg et al., 1990) We recently identified that PSA contains a glycolipid portion, which structurally resembles B. fragilis LOS, and now believe that the polysaccharide domain of PSA is covalently linked to LOS as a means of attachment to the OM. (Erturk-Hasdemir et al., 2019) The lipid A component of the LOS of Bacteroides has structural and biological features which distinguish it from the much studied lipid A of E. coli. (Erturk-Hasdemir et al., 2019; Kasper, 1976) It was therefore hypothesized that the LOS portion of PSA is responsible for induction of IFNβ. Consistent with this hypothesis, purified B. fragilis LOS was observed to induce IFNβ in a dose-dependent manner through TLR4 (FIGS. 4B-4C). In contrast, PSA that was delipidated using acid hydrolysis followed by chloroform/methanol extraction to remove the lipid induced no detectable levels of IFNβ (FIG. 4B), confirming that the LOS domain of PSA is responsible for IFNβ signaling.

### IFNβ Induction is a Shared Function of Bacteroides sp.

LOS-like molecules are not unique to B. fragilis, and multiple bacterial species of the genus Bacteroides have been demonstrated to have LPS or LOS molecules consisting of glycolipids that are structurally similar to B. fragilis LOS and distinct from classical E. coli LPS. (Cullen et al., 2015; Kasper, 1976; Vatanen et al., 2016) We therefore hypothesized that induction of IFNβ by Bacteroides LOS/LPS represents a broader mechanism by which commensal microbes are capable of influencing the host immune system. OM complexes were isolated from several different species of Bacteroides to test their effect on cytokine secretion by BMDCs. It was observed that OMs extracted from all of the Bacteroides sp. tested induced IFNβ by BMDCs in a TLR4-dependent manner (FIG. 4D), suggesting that the LPS/LOS species of each of these Bacteroides is indeed capable of signaling through TLR4 to induce IFNβ.

To address the role of Bacteroides sp. and TLR4 signaling in regulation of the IFN-I response in vivo, ISG expression was compared in WT SPF mice, Tlr4^{-/-} SPF mice, and WT SPF mice 7 days post treatment with metronidazole, a bactericidal antibiotic which specifically targets anaerobic bacteria, including Bacteroides sp. (Edwards, 1979; Salyers, 1984) WT mice treated with metronidazole alone and Tlr4^{-/-} SPF mice both exhibited significant decreases in ISG expression in the mLN (FIG. 4E, Table 3) and the spleen (FIG. 4F, Table 4). These findings confirm the potential for anaerobic commensal bacteria, of which Bacteroides are one of the most dominant taxa, to regulate the host IFN-I response through TLR4 signaling.

TABLE 3
Statistical analysis of mLN ISG expression. mLNs were harvested from age- and
gender-matched WT SPF mice with and without metronidazole (Met) treatment and
Tlr4^{-/-} SPF mice. RNA was isolated from whole tissue samples and qRT-PCR was
performed to analyze ISG expression levels. Fold change gene expression was
calculated compared to WT SPF mice using the delta-delta CT method, with ActB
as the reference gene.
Spleen fold difference gene expression to WT SPF

| | | | | WT SPF + Met | | | | Tlr4^{-/-} SPF | | | |
|---|---|---|---|---|---|---|---|---|---|---|---|
| | WT SPF | | | | | | p value to | | | | p value to |
| gene | Mean | SEM | N | Mean | SEM | N | WT SPF | Mean | SEM | N | WT SPF |
| Mx1 | 1.17 | 0.19 | 18 | 0.78 | 0.08 | 12 | 0.1179 (ns) | 0.74 | 0.14 | 8 | 0.1676 (ns) |
| Ifit1 | 1.09 | 0.13 | 15 | 0.57 | 0.13 | 8 | 0.0175* | 0.63 | 0.16 | 11 | 0.03* |
| Ifit2 | 1.04 | 0.08 | 18 | 0.69 | 0.07 | 12 | 0.0033** | 0.54 | 0.11 | 8 | 0.0011** |
| Ifit3 | 1.06 | 0.09 | 18 | 0.76 | 0.06 | 12 | 0.0273* | 1.01 | 0.14 | 8 | 0.7998 (ns) |
| Oas/2 | 1.02 | 0.05 | 18 | 0.74 | 0.07 | 12 | 0.002** | 0.93 | 0.06 | 8 | 0.2953 (ns) |
| Rsad2 | 1.11 | 0.12 | 16 | 0.46 | 0.09 | 12 | 0.0005*** | 0.35 | 0.05 | 7 | 0.0007*** |
| Irf7 | 1.02 | 0.06 | 13 | 0.68 | 0.07 | 12 | 0.0011** | 0.72 | 0.13 | 4 | 0.0322* |

N = number of samples analyzed,
SEM = standard error of the mean,
ns = not significant,
*p<0.05,
**p<0.01.

TABLE 4
Statistical analysis of spleen ISG expression. Spleens were harvested from age- and
gender-matched WT SPF mice with and without metronidazole (Met) treatment and
Tlr4^{-/-} SPF mice. RNA was isolated from whole tissue samples and qRT-PCR
was performed to analyze ISG expression levels. Fold change gene expression was
calculated compared to WT SPF mice using the delta-delta CT method, with
ActB as the reference gene.
Spleen fold difference gene expression to WT SPF

| | | | | WT SPF + Met | | | | Tlr4^{-/-} SPF | | | |
|---|---|---|---|---|---|---|---|---|---|---|---|
| | WT SPF | | | | | | p value to | | | | p value to |
| gene | Mean | SEM | N | Mean | SEM | N | WT SPF | Mean | SEM | N | WT SPF |
| Mx1 | 1.17 | 0.19 | 18 | 0.78 | 0.08 | 12 | 0.1179 (ns) | 0.74 | 0.14 | 8 | 0.1676 (ns) |
| Ifit1 | 1.09 | 0.13 | 15 | 0.57 | 0.13 | 8 | 0.0175* | 0.63 | 0.16 | 11 | 0.03* |
| Ifit2 | 1.04 | 0.08 | 18 | 0.69 | 0.07 | 12 | 0.0033** | 0.54 | 0.11 | 8 | 0.0011** |
| Ifit3 | 1.06 | 0.09 | 18 | 0.76 | 0.06 | 12 | 0.0273* | 1.01 | 0.14 | 8 | 0.7998 (ns) |
| Oas/2 | 1.02 | 0.05 | 18 | 0.74 | 0.07 | 12 | 0.002** | 0.93 | 0.06 | 8 | 0.2953 (ns) |
| Rsad2 | 1.11 | 0.12 | 16 | 0.46 | 0.09 | 12 | 0.0005*** | 0.35 | 0.05 | 7 | 0.0007*** |
| Irf7 | 1.02 | 0.06 | 13 | 0.68 | 0.07 | 12 | 0.0011** | 0.72 | 0.13 | 4 | 0.0322* |

N = number of samples analyzed,
SEM = standard error of the mean,
ns = not significant,
*p < 0.05,
**p < 0.01,
***p < 0.001.

### Commensal-Induced IFNβ Enhances Resistance to VSV Infection

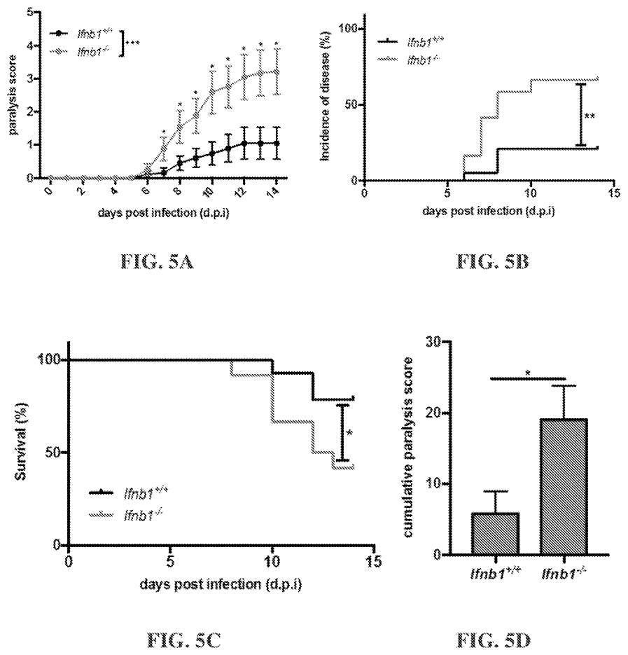

It was hypothesized that commensal-induced IFNβ is functionally important for the priming of the antiviral response and resistance to virus infection. A murine vesicular stomatitis virus (VSV) infection model, which causes meningoencephalitis and fatal paralytic disease, was used to investigate the role of commensal-induced IFNβ. To test the contribution of IFNβ to protection in this disease model, cohoused Ifnb1^{+/+} and Ifnb1^{-/-} littermates were infected by subcutaneous injection into the footpad with 10⁶ plaque-forming units (PFU) of VSV strain Indiana. Ifnb1^{-/-} mice exhibited increased susceptibility to disease based on all parameters tracked, with increased daily and cumulative disease scores (FIGS. 5A and 5D), incidence of disease (FIG. 5B), and ultimately reduced survival (FIG. 5C). These findings demonstrate a strong role for IFNβ in protection against subcutaneous VSV infection.

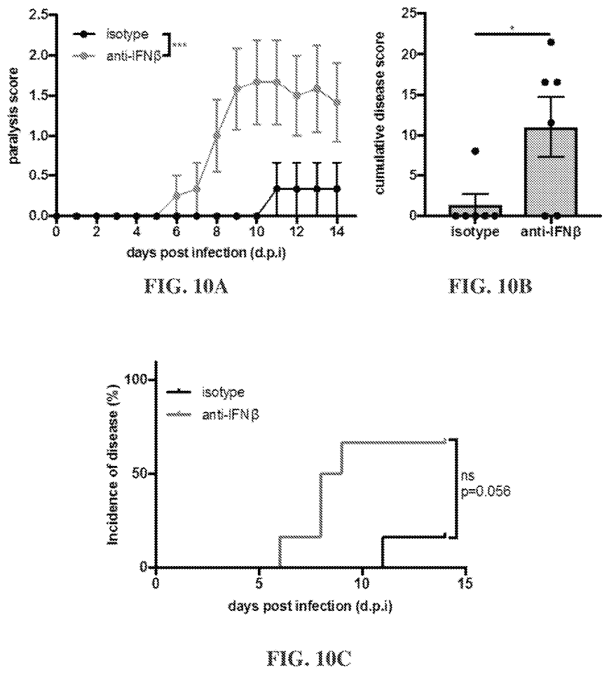

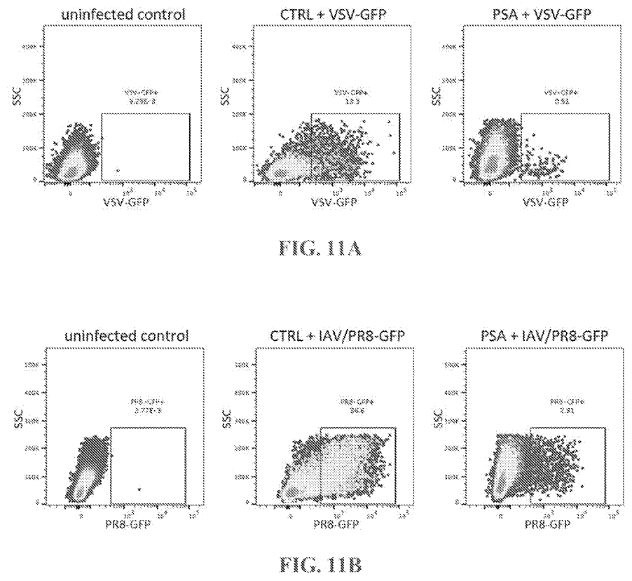

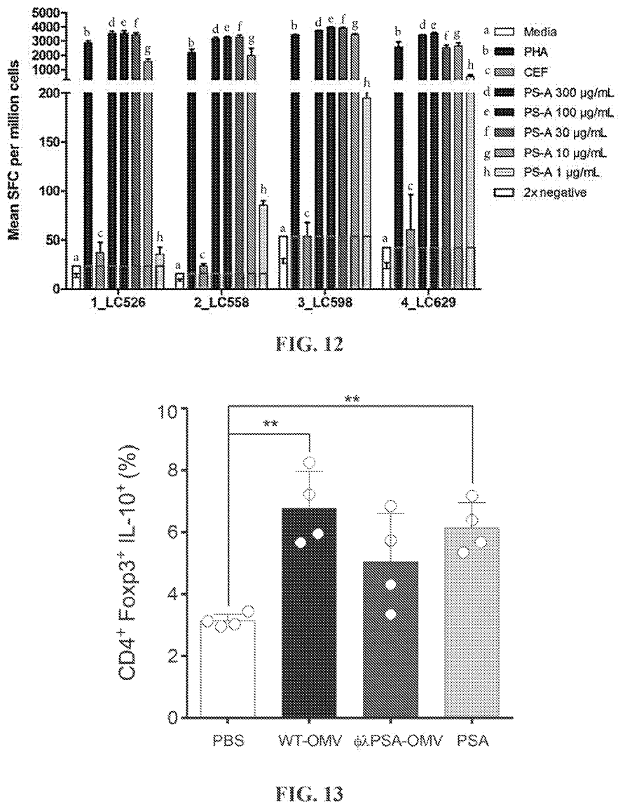

It is important to note that VSV infection itself induces IFNβ expression through PRR signaling, which likely contributes to ISG expression after infection and antiviral immunity. (Kato et al., 2005) Therefore, the increased susceptibility observed in the Ifnb1^{-/-} mice likely reflects virus infection-induced IFNβ in addition to any contributions of priming levels of IFNβ induced by commensal microbes prior to infection. To investigate the effects of priming of the antiviral response by constitutive, microbiota-induced IFNβ, an IFNβ neutralizing antibody (anti-IFNβ) was used to selectively inhibit IFNβ prior to infection. (Sheehan et al., 2015). WT mice were administered two doses of anti-IFNβ or mouse IgG2a isotype control antibody by IP injection, 72 hrs and 24 hrs prior to infection with 10⁶ PFU of VSV. Anti-IFNβ treated mice had increased severity of disease (FIGS. 10A-10C) compared to isotype control treated mice. These data reveal that blockade of IFNβ prior to infection enhances susceptibility to subsequent VSV infection, thus supporting a role for commensal-induced, homeostatic IFNβ.

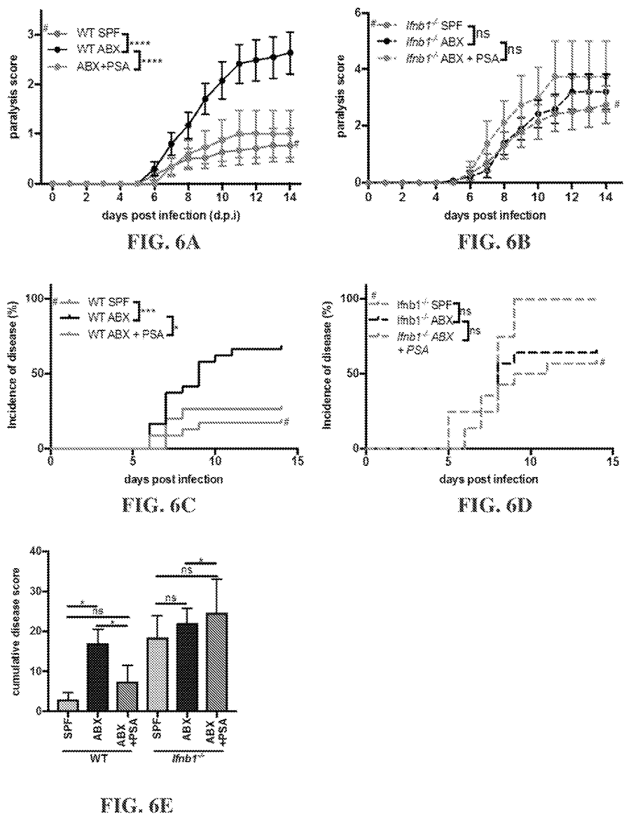

To further investigate the impact of the commensal microbiota on VSV infection, WT mice were administered broad-spectrum antibiotics for 7 days prior to infection, stopping on the day of infection. Increased susceptibility to disease was observed in WT ABX mice compared to WT SPF mice, with significantly increased paralysis scores (FIGS. 6A, 6G), incidence of disease (FIG. 6B), and reduced survival (FIG. 6C). These data reveal a novel role for the commensal microbiota in natural resistance to VSV infection. Ifnb1^{-/-} mice were also subjected to 7 days of antibiotics treatment prior to infection with VSV Indiana. Interestingly, antibiotics treatment had no effect on the susceptibility of Ifnb1^{-/-} mice to VSV infection (FIGS. 6D-6G). In the absence of IFNβ, depletion of the microbiota therefore does not alter the antiviral immune response to VSV, demonstrating that the microbiota exerts its protective effect specifically through IFNβ and that commensal-induced IFNβ is necessary for optimal resistance to virus infection.

B. fragilis PSA Possesses IFNβ-Mediated Antiviral Activity

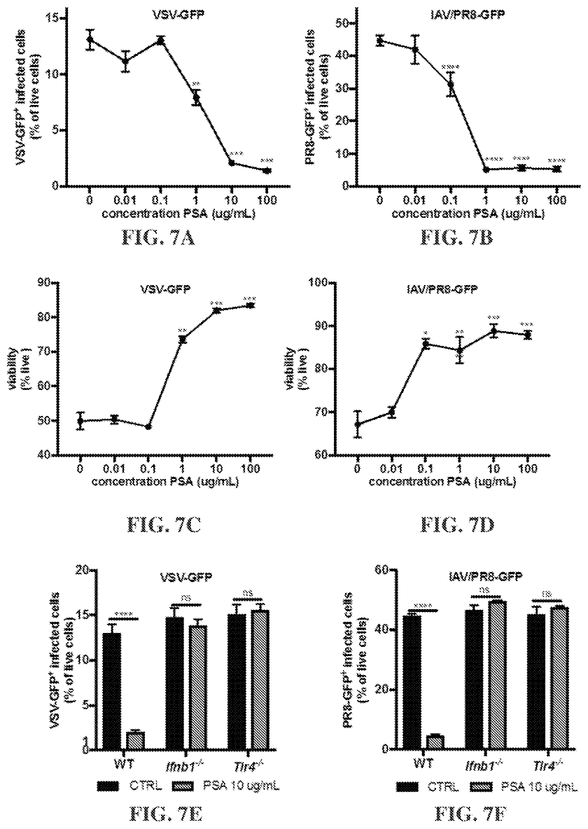

To further investigate the antiviral activity of commensal-induced IFNβ, we sought to identify whether signaling of a purified IFNβ-inducing commensal microbial molecule, B. fragilis PSA, is sufficient to protect against virus infection. While LOS and the LOS domain of PSA are responsible for IFNβ induction, whole PSA was used in this system due to its enhanced solubility. An in vitro virus infection model was developed in which BMDCs were treated with a dose response of PSA for 24 hrs prior to infection with a green fluorescent protein (GFP) expressing strain of VSV Indiana (VSV-GFP) at a multiplicity of infection (MOI) of 1, followed by flow cytometric analysis of the percentage of infected (GFP⁺) live cells 24 hrs post infection (h.p.i.) (FIG. 11A). Priming with PSA inhibited virus infection in a dose-dependent manner, resulting not only in a lower percentage of infected cells (FIG. 7A) but also increased cell viability (FIG. 7C). To investigate the mechanism of antiviral activity, infection with VSV-GFP was compared in WT, Ifnb1^{-/-}, or Tlr4^{-/-} BMDCs that were primed with PSA for 24 hrs. Protection by PSA was completely lost in the absence of TLR4 or IFNβ signaling (FIG. 7E), confirming that the observed antiviral function of PSA is indeed mediated by IFNβ signaling.

To identify whether the observed in vitro protective effects of PSA are limited to VSV infection, a second virus infection model was developed, using the murine adapted influenza A virus (IAV) strain PR8 (IAV/PR8). WT, or Tlr4^{-/-} BMDCs were primed with PSA followed by infection with a GFP-expressing IAV/PR8 strain (PR8-GFP) at an MOI of 1, followed by flow cytometric analysis 24 h.p.i. (FIG. 11B). Priming of WT BMDCs with PSA inhibited subsequent IAV infection, with a significant reduction in the percentage of infected cells (FIG. 7B) and increase in cell viability (FIG. 7D). This antiviral effect was completely abrogated in the absence of IFNβ or TLR4 (FIG. 7F), revealing that signaling of PSA through the same pathway protects cells from infection with two different viruses. These results establish a novel in vitro broad-spectrum antiviral function of B. fragilis PSA.

To investigate the in vivo antiviral activity of PSA, WT or Ifnb1^{-/-} ABX mice were administered PSA daily by oral gavage starting 4 days prior to and continuing to the day of infection with VSV. PSA treatment significantly reduced disease severity in WT ABX mice, with decreased daily and cumulative paralysis scores (FIGS. 6A, 6G), incidence of disease (FIG. 6B), and enhanced survival (FIG. 6C), to levels statistically indistinguishable from WT SPF mice. In Ifnb1^{-/-} ABX mice, PSA treatment had no observable effect on disease severity (FIGS. 6D-6G), supporting the hypothesis that the protective effects of PSA are mediated through IFNβ. These findings demonstrate that not only is commensal induction of IFNβ necessary for protection against VSV infection, but treatment with a single IFNβ-inducing commensal microbial molecule is sufficient to restore the protective effects of the whole microbiota in this model.

### Discussion

We have described a novel mechanism of immunomodulation by the commensal microbiota through IFNβ-mediated regulation of the homeostatic IFN-I response. Furthermore, we identified the mechanism by which a specific species of commensal bacteria regulates this response, signaling of B. fragilis glycolipids through TLR4 to induce expression of IFNβ by colon LP DCs. Importantly, this commensal-mediated regulation of IFNβ and the IFN-I response was found to play a critical role in maintaining health of the host by enhancing resistance to virus infection.

Our findings expand the analysis of ISG expression to multiple tissue sites as well as identifying which IFN-I family cytokine member is required for regulation of homeostatic ISG expression, a specific cellular source of commensal-induced IFNβ, and the molecular mechanism of IFNβ induction by a commensal microbial species. Indeed, ISG expression was reduced in Ifnb1^{-/-} mice, with no additional reduction upon antibiotics treatment, specifically confirming the role of commensal-induced IFNβ in the homeostatic IFN-I response. Interestingly, among the IFN-I family members, IFNβ has been demonstrated to possess unique functionality, with increased anti-proliferative and regulatory function. IFN-I signaling can result in several different signaling outcomes all mediated through the same receptor. Importantly, the many different biological outcomes, whether pro- or anti-inflammatory, could be either beneficial or detrimental to the host depending on the context. The observed specific induction of IFNβ by the microbiota suggests controlled expression of the distinct IFN-Is to favor homeostatic expression only of the more regulatory variant, IFNβ.

Colon LP DCs were identified as a major source of IFNβ, with lower levels of IFNβ expression also detected in mLN DCs but not in splenic DCs. Intestinal DCs have previously been established to carry commensal antigen to the mLN, where they are restricted from entering into systemic immune circulation, and thus remain in the intestinal immune compartment. (Macpherson and Uhr, 2004) Our results are consistent with a model in which IFNβ is expressed in response to detection of microbial antigens by colon LP DCs, which then remain in the intestinal LP or traffic to the mLN. Despite this tight locational restriction of IFNβ expression, we observed systemic decreases in ISG expression in both Ifnb1^{-/-} and antibiotics treated mice as well as functional effects of commensal induced-IFNβ during virus infection at an extra-intestinal site. It is possible that IFNβ secreted in the intestinal environment might enter the systemic circulation to mediate these effects. However, there were no detectable levels of IFNβ in the serum of WT SPF mice when analyzed by ELISA. IFNβ is extremely potent, capable of inducing ISG expression with a half-maximal effective concentration (EC₅₀) of 0.2 pM. (Schreiber and Piehler, 2015) It is therefore possible that even a minute quantity of IFNβ, below the limits of detection of ELISA, might enter the circulation from the intestinal immune compartment and induce systemic effects on ISG expression. Alternatively, secretion of IFNβ by intestinal DCs might induce gene expression changes locally, in neighboring cells, which can then traffic to distal body sites. Further investigation will be required to determine which of these mechanisms accounts for systemic ISG expression under homeostatic conditions.

B. fragilis is a Gram-negative human commensal microbe, residing primarily in the lower GI tract. (Mazmanian et al., 2005) We discovered that the glycolipids of B. fragilis, PSA and LOS, signal through TLR4 to induce expression of IFNβ by DCs. Canonically, activation of TLR4 by bacterial LPS signals not only through TRIF to induce IFN-I signaling, but also through MyD88 to induce proinflammatory mediators including tumor necrosis factor α (TNFα), inducible nitric oxide synthase (iNOS), and IL-12. (Maeshima and Fernandez, 2013) Through activation of a robust and uncontrolled immune response, LPS exposure can cause severe systemic inflammation, leading to sepsis and in some cases potentially lethal septic shock and multi-organ failure. (Bohannon et al., 2013; Molinaro et al., 2015) As a result of its endotoxicity and the associated adverse effects, the clinical applications of LPS as an immunomodulator have been limited. However, most research on LPS has focused on E. coli. (Maeshima and Fernandez, 2013) Using numerous assays including proinflammatory cytokine secretion, the Limulus amoebocyte lysate (LAL) assay, rabbit pyrogenicity test, chick embryo lethality, and the Swartzman reaction, B. fragilis LOS has previously been demonstrated to have low endotoxic activity, suggesting it is a less inflammatory LPS species. (Erturk-Hasdemir et al., 2019; Kasper, 1976; Sveen et al., 1977) This difference in activity can be attributed to several structural differences between the two molecules. Whereas E. coli LPS is hexa-acylated, B. fragilis LOS represents a mixture of penta-, tetra-, and tri-acylated molecules. (Weintraub et al., 1989) In addition, the acyl-chains of B. fragilis LOS are longer, with C15-C17 fatty acid chains compared to C12-C14 fatty acids used by E. coli LPS. (Raetz et al., 2007; Weintraub et al., 1989) Finally, the diglucosamine backbone of B. fragilis LOS is mono- or non-phosphorylated compared to the bis-phosphorylated lipid A of E. coli. (Molinaro et al., 2015; Weintraub et al., 1989) Numerous studies have revealed that the number and length of the acyl-chains as well as the phosphorylation state of the disaccharide backbone in LPS are critical factors affecting the interaction with TLR4 and the downstream function of these molecules. (Maeshima and Fernandez, 2013) In this way, B. fragilis glycolipids might be able to induce the protective beneficial effects of IFNβ without the consequences of uncontrolled, pathologic inflammation.

Our findings suggest that induction of IFNβ through TLR4 signaling is not limited to B. fragilis but is a shared function of an entire class of commensal microbial molecules, Bacteroides LPS/LOS. Indeed, OM extracts from all of the Bacteroides sp. tested were able to induce IFNβ. OMs comprise numerous bacterial molecules, all of which have the potential to interact with immune cells and influence their response. (Kasper and Seiler, 1975; May and Grabowicz, 2018) Isolation of LPS/LOS from each of these bacterial species will therefore be required to further assess their IFNβ-inducing capabilities. Importantly, the genus Bacteroides makes up a large proportion of the human GI microbiome and is widespread in prevalence across the human population. (Salyers, 1984; Wexler, 2007) Indeed, Bacteroides LOS/LPS species might be the most abundant microbial molecules in the GI tract. As such, it is plausible that Bacteroides-induced IFNβ represents a ubiquitous and crucial mechanism by which commensal microbes communicate with their host to regulate the immune system and other physiological processes, ultimately contributing to human health.

The observed antiviral effect of commensal-induced IFNβ is mediated through priming of constitutive expression of ISGs, thus arming the immune system to respond immediately and robustly to virus infection. Due to the important role of IFN-I signaling and ISG expression in the context of the majority of mammalian virus infections, such a finding might represent a broad-spectrum, and therefore universally important, antiviral activity of the commensal microbiota. Current IFN-I based therapies rely on the administration of non-physiologic amounts of exogenous IFN-Is, which can have unwanted side effects. (Gottberg et al., 2000; Sleijfer et al., 2005) Delivery of an IFNβ-inducing microbial molecule thus represents a novel IFN-I-based therapeutic approach, which could enhance the IFN-I response while still being subjected to homeostatic regulatory mechanisms, reducing the potential for undesired side effects.

### Experimental Procedures

### Mice

Conventional SPF mice of strains WT (C57BL/6, stock number 000664), Ifnar1^{-/-} (B6.129S2-Ifnar1^{tmIAgt}/Mmjax, stock number 010830), Tlr4^{-/-} (B6.B10ScN-Tlr4^{Ips-deI}/JthJ, stock number 007227), Myd88^{-/-} (B6.129P2(SJL)-Myd88^{tm1,1Defr}/J, stock number 009088), Trif^{-/-} (C57BL/6J-Ticam1^{LPs2}/L, stock number 005037) and IFNβ-YFP (B6.129-Ifnb1^{tm1Lky}/J, stock number 010818) on a C57BL/6 background were purchased from Jackson Laboratory. The Ifnb1^{-/-} (Ifnb1^{tm1(komp)vlcg}) mouse strain used for this research project was created from ES cell clone 12782A-D4, generated by Regeneron Pharmaceuticals, Inc. and obtained from the KOMP Repository (www.komp.org). Mice were housed under SPF conditions. All genetically deficient mice and their respective controls were gender- and age-matched (typically 5-10 weeks). All experiments on animals were approved by the Harvard Medical Area Standing Committee on Animals (animal protocol number IS00000187-3). Germ free C57BL/6 mice were bred and maintained in sterile vinyl isolators in the animal facility at Harvard Medical School.

### Antibiotics Treatment

For studies involving depletion of the microbiota, SPF mice of the indicated strains were treated with a broad-spectrum antibiotics cocktail for 7 days. Vancomycin hydrochloride (0.5 g/L, Alfa Aesar), gentamicin sulfate (0.5 g/L, Sigma-Aldrich), and ampicillin (1 g/L, MP Biomedicals) were administered in the drinking water. Metronidazole (8 g/L, Alfa Aesar) and amphotericin B (0.1 g/L, Amresco Inc) were administered in 200 uL by OG once daily. Fecal samples were collected and plated under aerobic and anaerobic conditions to confirm microbiota depletion. For studies involving treatment with metronidazole alone, Metronidazole (0.5 g/L, Alfa Aesar) was administered in the drinking water for 7 days.

### Bacteria Culture

All bacteria used in this study were grown on Brucella blood agar plates or in liquid culture in rich Brain Heart Infusion Broth (BD) supplemented with Hemin (0.01%), vitamin K (0.1 mg/mL), glucose (0.5%), Monopotassium phosphate (0.5%), and L-cysteine (0.1%) in an anaerobic chamber (Coy Industries) at 37° Celsius (C). The following bacteria were used in this study B. fragilis (strain NCTC 9343, ATCC 25285), B. thetaiotaomicron (B. theta, ATCC 29741), B. vulgatus (ATCC 8482), B. dorei (DSM 17855), B. uniformis (ATCC 8492), B. ovatus (ATCC 8483), and C. ramosum (strain A031).

qRT-PCR Gene Expression Analysis

RNA was isolated from whole tissues or the DC⁺ and DC⁻ fractions of spleens, mLNs, and colons following manufacturer's instructions with the RNeasy mini kit (Qiagen). cDNA was prepared with the Quantitect Reverse Transcription Kit (Qiagen). qRT-PCR was performed on a QuantStudio7 Flex or Pro Real-Time PCR system (Applied Biosystems) with RT² SYBR Green ROX qPCR Mastermix (Qiagen). Amplification was achieved using an initial cycle of 95° C. for 10 min, followed by 40 cycles of 95° C. for 15 s and 60° C. for 1 min. Gene expression was normalized using Actb as a reference gene. The forward (f) and reverse (r) primers used to detect expression of the corresponding murine genes were as follows (SEQ ID NOs: 1-20, respectively):

Ifit1
(f 5′-CAGAAGCACACATTGAAGAA-3′,
r 5′-TGTAAGTAGCCAGAGGAAGG-3′),
Ifit2
(5′-GGGAAAGCAGAGGAAATCAA-3′,
r 5′-TGAAAGTTGCCATACAGAAG-3′),
Ifit3
(f 5′-GCCGTTACAGGGAAATACTGG-3′,
r 5′-CCTCAACATCGGGGCTCT-3′),
Oasl2
(f 5′-GGATGCCTGGGAGAGAATCG-3′,
r 5′-TCGCCTGCTCTTCGAAACTG-3′),
Rsad2
(f 5′-AACAGGCTGGTTTGGAGAAG-3′,
r: 5′-TGCCATTGCTCACTATGCTC-3′),
Irf7
(f 5′-GCCAGGAGCAAGACCGTGTT-3′,
r 5′-TGCCCCACCACTGCCTGTA-3′),
Gbp4
(f 5′-TGGGGGACACAGGCTCTACA-3′,
r 5′-GCCTGCAGGATGGAACTCTCAA-3′),
ifnb1
(f 5′-CCTACAGGGCGGACTTCAAG-3′,
r 5′-GGATGGCAAAGGCAGTGTAACT-3′),
Bst2
(f 5′-GAAGTCACGAAGCTGAACCA-3′,
r 5′ -CCTGCACTGTGCTAGAAGTCTC-3′),
Actb
(f 5′-GATGCTCCCCGGGCTGTATT-3′,
r 5′-GGGGTACTTCAGGGTCAGGA-3′).

### Preparation of Tissue Single-Cell Suspensions

Single-cell suspensions of the colonic lamina propria were prepared as previously described. (Dasgupta et al., 2014) Briefly, the tissue was cut transversely into approximately 2 cm pieces and then longitudinally to expose the lumen. To extract epithelial cells, tissue pieces were incubated in 1 mM dithiothreitol (Sigma) in PBS for 10 minutes, washed, and incubated in 30 mM Ethylenediaminetetraacetic acid (EDTA) three times for 8 minutes each. To digest the tissue, colon pieces were treated with RPMI medium supplemented with 5% fetal bovine serum (FBS) and 1 mg/mL collagenase type IV for 45 minutes at 37° C. in an atmosphere of 5% carbon dioxide (CO₂) and then passed through a 70 μM filter. For single-cell suspensions of mLNs and spleens, tissues were harvested, treated with 1 mg/mL collagenase type IV in RPMI for 30 minutes at 37° C. in an atmosphere of 5% CO₂, and passed through a 70 μm filter. Splenic samples were treated with red blood cell lysis buffer (Gibco).

### Dendritic Cell Isolation

Single-cell suspensions of the indicated tissues were prepared as described. DC positive and negative fractions were isolated with mouse pan-DC microbeads (Miltenyi), following the manufacturer's protocol.

### Monocolonization of GF Mice

GFWT C57BL/6 mice were administered B. fragilis strain NCTC 9343 or C. ramosum strain AO31 by oral gavage [200 uL, approximately 10⁸-10⁹ colony-forming units (CFU) per mouse]. Mice were housed in autoclaved cages and maintained on a diet of autoclaved food and water for 2 weeks and then euthanized for analysis.

### IFNβ ELISA Analysis

For serum analysis, WT SPF or antibiotics treated mice were euthanized and blood was collected into 1.1 ml Z-Gel Micro Tubes (Sarstedt). Blood was left at room temperature for 30 minutes, centrifuged to remove coagulated cells [12,000 revolutions per minute (RPM), 10 minutes], and levels of IFNβ in the serum were quantified with the High Sensitivity Mouse IFN Beta ELISA kit (PBL Assay Science) according to the manufacturer's protocol. For cell culture supernatant analysis, cell free supernatants were collected from BMDC cultures and IFNβ levels were quantified with the Mouse IFN Beta ELISA kit (PBL Assay Science) according to the manufacturer's protocol.

### BMDC Culture

DCs were derived from bone marrow of the indicated mice as previously described. (Helft et al., 2015) Briefly, bone marrow was collected from femurs, treated with red blood cell lysis buffer (Gibco), and cultured in 10 cm tissue culture (TC)-treated dishes at a density of 10×10⁶ cells in 10 mL per plate in complete RPMI medium (cRPMI: RPMI 1640 supplemented with 10 mM HEPES, 10% fetal bovine serum, 2 mM L-glutamine, 50 units/mL penicillin, 50 μg/mL streptomycin, and 50 μM 2-mercaptoethanol) with 20 ng/mL granulocyte-macrophage colony-stimulating factor (GM-CSF). Cells were cultured for 6 days at 37° C. in an atmosphere of 5% CO₂ with replenishment of medium on day 3. On day 6 the non-adherent and loosely adherent cells were harvested and plated at a density of 10⁶ cells/mL in 200 uL per well of a 96-well round-bottom TC-treated cell culture plate for use in the indicated experimental procedures.

### Bacteroides OM Extraction

OM complexes were isolated from the indicated Bacteroides sp. as previously described. (Kasper and Seiler, 1975) Briefly, mid-log phase bacteria were pelleted, washed twice in 0.15 M sodium chloride (NaCl) and resuspended in a buffer of 0.15 M NaCl, 0.05 M sodium phosphate, and 0.01 M EDTA. The resuspended organisms were incubated for 30 minutes at 60° C. then subjected to mild shearing pressure by passing through a 25-gauge hypodermic needle. Cells were pelleted from the suspension and the supernatants were harvested and subjected to two rounds of centrifugation at 33,000 RPM for 2 hrs at 4° C. to pellet the OM extracts.

B. fragilis Glycolipid and Polysaccharide Purification

PSA was purified from B. fragilis mutant strain 444 and LOS was purified from an acapsular B. fragilis mutant strain by phenol-water extraction as previously described. (Tzianabos et al., 1992) To obtain the purified delipidated polysaccharide domain of PSA, PSA was subjected to acid hydrolysis with 2% acetic acid at 90° C. for 2 hours. The sample was neutralized the lipid was removed using chloroform/methanol extraction.

### Flow Cytometric Analysis

The following mouse-specific IgG monoclonal antibodies were purchased from Biolegend: Pacific Blue-conjugated anti-CD45, PE-conjugated anti-CX3CR1, PerCP-Cy5.5 conjugated anti-CD11b, PE-Cy7-conjugated anti-CD11c, and APC-conjugated anti-CD103. Fixable Viability Dye eFluor780 was purchased from eBioscience. Single-cell suspensions were stained with a suitable combination of fluorochrome-conjugated antibodies and fixable viability dye. Cells were fixed in 2% paraformaldehyde in PBS, and examined with an LSR II flow cytometer (BD). The data were analyzed with FlowJo software.

### Mouse VSV Infection

Mice were anesthetized by IP injection of ketamine and xylazine. 1×10⁶ PFU of VSV strain Indiana was delivered in 10 uL per mouse by subcutaneous injection into the footpad as previously described. (Fensterl et al., 2014) Body weight and clinical disease score were monitored daily for 14 days following infection. VSV-infected mice exhibit an ascending paralytic disease that was scored as follows: 0=no symptoms, 1=tail paralysis, 2=hind-limb weakness, 2.5=paralysis of one hind limb, 3=paralysis of both hind limbs, 3.5=paralysis of both hind limbs and forelimb weakness, 4=forelimb and hind-limb paralysis, 5=moribund). Mice with a body condition score ≤2 or a disease score ≥4 were humanely euthanized.

### In Vitro Virus Infections

On day 6 of culture, GM-CSF BMDCs were plated at a density of 10⁶ cells/mL in 200 uL of cRPMI medium per well of a 96-well round-bottom TC-treated cell culture plate with 20 ng/mL GM-CSF and the indicated treatments for 24 hrs, washed, and resuspended in cRPMI medium containing either IAV/PR8-GFP or VSV-GFP at an MOI of 1. Cells were harvested for flow cytometric analysis 24 hrs post infection.

IFNβ neutralization

Mice were administered two doses of 250 ug IFNβ neutralizing antibody (anti-IFNβ, clone HDβ-4A7; Leinco Technologies) or Mouse IgG2a isotype control antibody (Leinco Technologies) by IP injection, 72 hrs and 24 hrs prior to infection with 10⁶ PFU of VSV strain Indiana.

### Statistical Analysis

Information regarding statistical details and methods for each experiment can be found in the figure legends. Data is presented as mean +/− SEM unless otherwise indicated. Statistical analysis was performed using GraphPad Prism 7.0.

### REFERENCES

Abt, M. C., Osborne, L. C., Monticelli, L. A., Doering, T. A., Alenghat, T., Sonnenberg, G. F., Paley, M. A., Antenus, M., Williams, K. L., Erikson, J., et al. (2012). Commensal bacteria calibrate the activation threshold of innate antiviral immunity. Immunity 37, 158-170.
An, D., Oh, S. F., Olszak, T., Neves, J. F., Avci, F. Y., Erturk-Hasdemir, D., Lu, X., Zeissig, S., Blumberg, R. S., and Kasper, D. L. (2014). Sphingolipids from a symbiotic microbe regulate homeostasis of host intestinal natural killer T cells. Cell 156, 123-133.
Bohannon, J. K., Hernandez, A., Enkhbaatar, P., Adams, W. L., and Sherwood, E. R. (2013). The immunobiology of toll-like receptor 4 agonists: from endotoxin tolerance to immunoadjuvants. Shock 40, 451-462.
Chirdo, F. G., Millington, O. R., Beacock-Sharp, H., and Mowat, A. M. (2005). Immunomodulatory dendritic cells in intestinal lamina propria. Eur J Immunol 35, 1831-1840.
Cress, B. F., Englaender, J. A., He, W., Kasper, D., Linhardt, R. J., and Koffas, M. A. (2014). Masquerading microbial pathogens: capsular polysaccharides mimic host-tissue molecules. FEMS Microbiol Rev 38, 660-697.
Cullen, T. W., Schofield, W. B., Barry, N. A., Putnam, E. E., Rundell, E. A., Trent, M. S., Degnan, P. H., Booth, C. J., Yu, H., and Goodman, A. L. (2015). Gut microbiota. Antimicrobial peptide resistance mediates resilience of prominent gut commensals during inflammation. Science 347, 170-175.
Dalton, K. P., and Rose, J. K. (2001). Vesicular stomatitis virus glycoprotein containing the entire green fluorescent protein on its cytoplasmic domain is incorporated efficiently into virus particles. Virology 279, 414-421.
Dasgupta, S., Erturk-Hasdemir, D., Ochoa-Reparaz, J., Reinecker, H. C., and Kasper, D. L. (2014). Plasmacytoid dendritic cells mediate anti-inflammatory responses to a gut commensal molecule via both innate and adaptive mechanisms. Cell Host Microbe 15, 413-423.
Edwards, D. I. (1979). Mechanism of antimicrobial action of metronidazole. J Antimicrob Chemother 5, 499-502.
Erturk-Hasdemir, D., and Kasper, D. L. (2013). Resident commensals shaping immunity. Curr Opin Immunol 25, 450-455.
Erturk-Hasdemir, D., Oh, S. F., Okan, N. A., Stefanetti, G., Gazzaniga, F. S., Seeberger, P. H., Plevy, S. E., and Kasper, D. L. (2019). Symbionts exploit complex signaling to educate the immune system. Proc Natl Acad Sci USA.
Fensterl, V., Wetzel, J. L., and Sen, G. C. (2014). Interferon-induced protein Ifit2 protects mice from infection of the peripheral nervous system by vesicular stomatitis virus. J Virol 88, 10303-10311.
Geva-Zatorsky, N., Sefik, E., Kua, L., Pasman, L., Tan, T. G., Ortiz-Lopez, A., Yanortsang, T. B., Yang, L., Jupp, R., Mathis, D., et al. (2017). Mining the Human Gut Microbiota for Immunomodulatory Organisms. Cell 168, 928-943 e911.
Gottberg, K., Gardulf, A., and Fredrikson, S. (2000). Interferon-beta treatment for patients with multiple sclerosis: the patients' perceptions of the side-effects. Mult Scler 6, 349-354.
Gough, D. J., Messina, N. L., Clarke, C. J., Johnstone, R. W., and Levy, D. E. (2012). Constitutive type I interferon modulates homeostatic balance through tonic signaling. Immunity 36, 166-174.
Helft, J., Bottcher, J., Chakravarty, P., Zelenay, S., Huotari, J., Schraml, B. U., Goubau, D., and Reis e Sousa, C. (2015). GM-CSF Mouse Bone Marrow Cultures Comprise a Heterogeneous Population of CD11c(+)MHCII(+) Macrophages and Dendritic Cells. Immunity 42, 1197-1211.
Human Microbiome Project, C. (2012). Structure, function and diversity of the healthy human microbiome. Nature 486, 207-214.
Ivashkiv, L. B., and Donlin, L. T. (2014). Regulation of type I interferon responses. Nat Rev Immunol 14, 36-49.
Kasper, D. L. (1976). Chemical and biological characterization of the lipopolysaccharide of Bacteroides fragilis subspecies fragilis. J Infect Dis 134, 59-66.
Kasper, D. L., and Seiler, M. W. (1975). Immunochemical characterization of the outer membrane complex of Bacteroides fragilis subspecies fragilis. J Infect Dis 132, 440-450.
Kato, H., Sato, S., Yoneyama, M., Yamamoto, M., Uematsu, S., Matsui, K., Tsujimura, T., Takeda, K., Fujita, T., Takeuchi, O., et al. (2005). Cell type-specific involvement of RIG-I in antiviral response. Immunity 23, 19-28.
Kole, A., He, J., Rivollier, A., Silveira, D. D., Kitamura, K., Maloy, K. J., and Kelsall, B. L. (2013). Type I IFNs regulate effector and regulatory T cell accumulation and anti-inflammatory cytokine production during T cell-mediated colitis. J Immunol 191, 2771-2779.
Lin, C. S., Chang, C. J., Lu, C. C., Martel, J., Ojcius, D. M., Ko, Y. F., Young, J. D., and Lai, H. C. (2014). Impact of the gut microbiota, prebiotics, and probiotics on human health and disease. Biomed J 37, 259-268.
Lindberg, A. A., Weintraub, A., Zahringer, U., and Rietschel, E. T. (1990). Structure-activity relationships in lipopolysaccharides of Bacteroides fragilis. Rev Infect Dis 12 Suppl 2, S133-141.
Lu, Y. C., Yeh, W. C., and Ohashi, P. S. (2008). LPS/TLR4 signal transduction pathway. Cytokine 42, 145-151.
Macpherson, A. J., and Uhr, T. (2004). Induction of protective IgA by intestinal dendritic cells carrying commensal bacteria. Science 303, 1662-1665.
Maeshima, N., and Fernandez, R. C. (2013). Recognition of lipid A variants by the TLR4-MD-2 receptor complex. Front Cell Infect Microbiol 3, 3.
Manicassamy, B., Manicassamy, S., Belicha-Villanueva, A., Pisanelli, G., Pulendran, B., and Garcia-Sastre, A. (2010). Analysis of in vivo dynamics of influenza virus infection in mice using a GFP reporter virus. Proc Natl Acad Sci USA 107, 11531-11536.
May, K. L., and Grabowicz, M. (2018). The bacterial outer membrane is an evolving antibiotic barrier. Proc Natl Acad Sci USA 115, 8852-8854.
Mazmanian, S. K., Liu, C. H., Tzianabos, A. O., and Kasper, D. L. (2005). An immunomodulatory molecule of symbiotic bacteria directs maturation of the host immune system. Cell 122, 107-118.
Mazmanian, S. K., Round, J. L., and Kasper, D. L. (2008). A microbial symbiosis factor prevents intestinal inflammatory disease. Nature 453, 620-625.
McNab, F., Mayer-Barber, K., Sher, A., Wack, A., and O'Garra, A. (2015). Type I interferons in infectious disease. Nat Rev Immunol 15, 87-103.
Molinaro, A., Holst, O., Di Lorenzo, F., Callaghan, M., Nurisso, A., D'Errico, G., Zamyatina, A., Peri, F., Berisio, R., Jerala, R., et al. (2015). Chemistry of lipid A: at the heart of innate immunity. Chemistry (Easton) 21, 500-519.
Morgun, A., Dzutsev, A., Dong, X., Greer, R. L., Sexton, D. J., Ravel, J., Schuster, M., Hsiao, W., Matzinger, P., and Shulzhenko, N. (2015). Uncovering effects of antibiotics on the host and microbiota using transkingdom gene networks. Gut 64, 1732-1743.
Muller, U., Steinhoff, U., Reis, L. F., Hemmi, S., Pavlovic, J., Zinkernagel, R. M., and Aguet, M. (1994). Functional role of type I and type II interferons in antiviral defense. Science 264, 1918-1921.
Qin, J., Li, R., Raes, J., Arumugam, M., Burgdorf, K. S., Manichanh, C., Nielsen, T., Pons, N., Levenez, F., Yamada, T., et al. (2010). A human gut microbial gene catalogue established by metagenomic sequencing. Nature 464, 59-65.
Raetz, C. R., Reynolds, C. M., Trent, M. S., and Bishop, R. E. (2007). Lipid A modification systems in gram-negative bacteria. Annu Rev Biochem 76, 295-329.
Round, J. L., and Mazmanian, S. K. (2010). Inducible Foxp3⁺ regulatory T-cell development by a commensal bacterium of the intestinal microbiota. Proc Natl Acad Sci USA 107.
Salyers, A. A. (1984). Bacteroides of the human lower intestinal tract. Annu Rev Microbiol 38, 293-313.
Savage, D. C. (1977). MICROBIAL ECOLOGY OF THE GASTROINTESTINAL TRACT. Annu Rev Microbiol 31, 107-133.
Schaupp, L., Muth, S., Rogell, L., Kofoed-Branzk, M., Melchior, F., Lienenklaus, S., Ganal-Vonarburg, S. C., Klein, M., Guendel, F., Hain, T., et al. (2020). Microbiota-Induced Type I Interferons Instruct a Poised Basal State of Dendritic Cells. Cell 181, 1080-1096 e1019.
Scheu, S., Dresing, P., and Locksley, R. M. (2008). Visualization of IFNbeta production by plasmacytoid versus conventional dendritic cells under specific stimulation conditions in vivo. Proc Natl Acad Sci USA 105, 20416-20421.
Schreiber, G., and Piehler, J. (2015). The molecular basis for functional plasticity in type I interferon signaling. Trends Immunol 36, 139-149.
Sefik, E., Geva-Zatorsky, N., Oh, S., Konnikova, L., Zemmour, D., McGuire, A. M., Burzyn, D., Ortiz-Lopez, A., Lobera, M., Yang, J., et al. (2015). MUCOSAL IMMUNOLOGY. Individual intestinal symbionts induce a distinct population of RORgamma(+) regulatory T cells. Science 349, 993-997.
Sender, R., Fuchs, S., and Milo, R. (2016). Are We Really Vastly Outnumbered? Revisiting the Ratio of Bacterial to Host Cells in Humans. Cell 164, 337-340.
Sheehan, K. C., Lazear, H. M., Diamond, M. S., and Schreiber, R. D. (2015). Selective Blockade of Interferon-alpha and -beta Reveals Their Non-Redundant Functions in a Mouse Model of West Nile Virus Infection. PLoS One 10, e0128636.
Sheikh, F., Dickensheets, H., Gamero, A. M., Vogel, S. N., and Donnelly, R. P. (2014). An essential role for IFN-beta in the induction of IFN-stimulated gene expression by LPS in macrophages. J Leukoc Biol 96, 591-600.
Singh-Jasuja, H., Thiolat, A., Ribon, M., Boissier, M. C., Bessis, N., Rammensee, H. G., and Decker, P. (2013). The mouse dendritic cell marker CD11c is down-regulated upon cell activation through Toll-like receptor triggering. Immunobiology 218, 28-39.
Sleijfer, S., Bannink, M., Van Gool, A. R., Kruit, W. H., and Stoter, G. (2005). Side effects of interferon-alpha therapy. Pharm World Sci 27, 423-431.
Stagg, A. J. (2018). Intestinal Dendritic Cells in Health and Gut Inflammation. Front Immunol 9, 2883.
Stetson, D. B., and Medzhitov, R. (2006). Type I interferons in host defense. Immunity 25, 373-381.
Surana, N. K., and Kasper, D. L. (2014). Deciphering the tete-a-tete between the microbiota and the immune system. J Clin Invest 124, 4197-4203.
Sveen, K., Hofstad, T., and Milner, K. C. (1977). Lethality for mice and chick embryos, pyrogenicity in rabbits and ability to gelate lysate from amoebocytes of Limulus polyphemus by lipopolysaccharides from Bacteroides, Fusobacterium and Veillonella. Acta Pathol Microbiol Scand B 85B, 388-396.
Theofilopoulos, A. N., Baccala, R., Beutler, B., and Kono, D. H. (2005). Type I interferons (alpha/beta) in immunity and autoimmunity. Annu Rev Immunol 23, 307-336.
Turnbaugh, P. J., Ley, R. E., Hamady, M., Fraser-Liggett, C. M., Knight, R., and Gordon, J. I. (2007). The human microbiome project. Nature 449, 804-810.
Tzianabos, A. O., Pantosti, A., Baumann, H., Brisson, J. R., Jennings, H. J., and Kasper, D. L. (1992). The capsular polysaccharide of Bacteroides fragilis comprises two ionically linked polysaccharides. J Biol Chem 267, 18230-18235.
van den Broek, M. F., Muller, U., Huang, S., Zinkernagel, R. M., and Aguet, M. (1995). Immune defence in mice lacking type I and/or type II interferon receptors. Immunol Rev 148, 5-18.
Vatanen, T., Kostic, A. D., d′Hennezel, E., Siljander, H., Franzosa, E. A., Yas sour, M., Kolde, R., Vlamakis, H., Arthur, T. D., Hamalainen, A. M., et al. (2016). Variation in Microbiome LPS Immunogenicity Contributes to Autoimmunity in Humans. Cell 165, 842-853.
Weintraub, A., Zahringer, U., Wollenweber, H. W., Seydel, U., and Rietschel, E. T. (1989). Structural characterization of the lipid A component of Bacteroides fragilis strain NCTC 9343 lipopolysaccharide. Eur J Biochem 183, 425-431.
Wexler, H. M. (2007). Bacteroides: the good, the bad, and the nitty-gritty. Clin Microbiol Rev 20, 593-621.
Yan, N., and Chen, Z. J. (2012). Intrinsic antiviral immunity. Nat Immunol 13, 214-222.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method for stimulating an anti-viral immune response in a subject, comprising administering to the subject an effective amount of a Bacteroides glycolipid comprising a diglucosamine substituted with one or more acyl chains, on a regular basis, wherein the immune response protects the subject against a viral infection resulting from exposure to a virus.

2. A method for treating a subject to protect against a viral infection resulting from exposure to a virus, comprising administering to the subject an effective amount of a Bacteroides glycolipid comprising a diglucosamine substituted with one or more acyl chains, on a regular basis.

3. A method for treating a subject, comprising administering an effective amount of a Bacteroides glycolipid comprising a diglucosamine substituted with one or more acyl chains, on a regular basis, to a subject at risk of exposure to a virus or developing a viral infection.

4. The method of claim 1, wherein a regular basis is daily, every two days, every 3 days, every 4 days, every 5 days, every 6 days, weekly, every two weeks, or monthly.

5. The method of claim 1, wherein the subject is immunocompromised.

6. The method of claim 1, wherein the subject was recently treated with an antibiotic, optionally an antibiotic that targets anaerobic bacteria, or an antibiotic that is a broad spectrum antibiotic.

7. The method of claim 6, wherein the subject was treated with the antibiotic in the last 2 months, in the last 1 month, or in the last week.

8. The method of claim 1, wherein the subject is being treated with an antibiotic, optionally an antibiotic that targets anaerobic bacteria.

9. The method of claim 1, wherein the subject will be treated with an antibiotic, optionally an antibiotic that targets anaerobic bacteria.

10. The method of claim 9, wherein the subject will be treated with an antibiotic in the next day, in the next week, or in the next month.

11. The method of claim 1, wherein the subject has come into contact with a subject exposed to the virus or having the viral infection or with a bodily fluid of a subject exposed to the virus or having the viral infection.

12. The method of claim 1, wherein the viral infection is selected from the group consisting of SARS coronavirus, influenza virus, Ebola virus, norovirus, Zika virus, West Nile virus, Dengue virus, herpesvirus, equine encephalitis virus, respiratory syncytial virus (RSV), cytomegalovirus, rabies virus, or measles virus infection.

13. (canceled)

14. The method of claim 1, wherein the Bacteroides glycolipid is provided in an unconjugated, cell-free and membrane-free form.

15. The method of claim 1, wherein the Bacteroides glycolipid is provided as lipidated PSA.

16. The method of claim 1, wherein the Bacteroides glycolipid is provided as Bacteroides outer membrane (OM).

17. The method of claim 1, wherein the Bacteroides glycolipid is provided as: (i) inactivated Bacteroides spp. or (ii) live attenuated Bacteroides spp.

18-19. (canceled)

20. A method for modulating TLR4 signaling comprising administering to a subject in need thereof an effective amount of a Bacteroides glycolipid comprising a diglucosamine substituted with one or more acyl chains.

21-22. (canceled)

23. The method of claim 20, wherein the subject is administered or exposed to a TLR4 agonist or TLR4 antagonist.

24. The method of claim 23, wherein the TLR4 agonist is buprenorphine, carbamazepine, fentanyl, levorphanol, lipopolysaccharide (LPS), methadone, morphine, oxycarbezepine, oxycodone, pethidine, tapentadol, or morphine-3-glucuronide.

25. (canceled)

26. The method of claim 25, wherein the TLR4 antagonist is naloxone, naltrexone, propentofylline, palmitoylethanolamide, amitriptyline, cyclobenzaprine, ketotifen, imipramine, mianserin, ibudilast, pinocembrin, or resatorvid.
